(19) United States
(12) Patent Application Publication
(10) Pub. No.: US 2019/0275030 A1
(43) Pub. Date: Sep. 12, 2019

(54) PHARMACEUTICAL COMPOSITIONS COMPRISING DEXTROMETHORPHAN AND QUINIDINE FOR THE TREATMENT OF DEPRESSION, ANXIETY, AND NEURODEGENERATIVE DISORDERS

(71) Applicant: Avanir Pharmaceuticals, Inc., Aliso Viejo, CA (US)

(72) Inventor: James Berg, San Diego, CA (US)

(21) Appl. No.: 16/157,136

(22) Filed: Oct. 11, 2018

Related U.S. Application Data
(63) Continuation of application No. 15/438,796, filed on Feb. 22, 2017, now abandoned.
Application No. 15/438,796 is a continuation of application No. 14/863,020, filed on Sep. 23, 2015, now abandoned.
Application No. 14/863,020 is a continuation of application No. 13/750,067, filed on Jan. 25, 2013, now abandoned.
Application No. 13/750,067 is a continuation of application No. 12/820,912, filed on Jun. 22, 2010, now abandoned.
Application No. 12/820,912 is a continuation of application No. 12/181,962, filed on Jul. 29, 2008, now abandoned.
Application No. 12/181,962 is a continuation of application No. PCT/US2007/002931, filed on Feb. 1, 2007.
(60) Provisional application No. 60/765,250, filed on Feb. 3, 2006.
(60) Provisional application No. 60/854,666, filed on Oct. 26, 2006.
(60) Provisional application No. 60/854,748, filed on Oct. 27, 2006.

(51) Int. Cl. A61K 31/49; A61K 31/485; A61K 45/06

(52) U.S. Cl. CPC A61K 31/49; A61K 45/06; A61K 31/485

(57) ABSTRACT

Pharmaceutical compositions and methods for treating depression, anxiety, and neurodegenerative diseases and cognitive disorders, such as dementia and Alzheimer's disease, by administering same are provided. The compositions comprise dextromethorphan in combination with quinidine.

### CROSS-REFERENCE TO RELATED APPLICATIONS

This application is a continuation of U.S. application Ser. No. 13/750,067, filed Jan. 25, 2013, which is a continuation of U.S. application Ser. No. 12/820,912, filed Jun. 22, 2010, which is a continuation of U.S. application Ser. No. 12/181,962, filed Jul. 29, 2008, which is a continuation, under 35 U.S.C. § 120, of International Patent Application No. PCT/US2007/002931, filed on Feb. 1, 2007 under the Patent Cooperation Treaty (PCT), which was published by the International Bureau in English on Aug. 16, 2007, which designates the United States and claims the benefit of U.S. Provisional Application No. 60/765,250, filed Feb. 3, 2006, U.S. Provisional Application No. 60/854,666, filed Oct. 26, 2006, and U.S. Provisional Application No. 60/854,748, filed Oct. 27, 2006, the disclosures of which are hereby expressly incorporated by reference in their entirety and are hereby expressly made a portion of this application.

### FIELD OF THE INVENTION

Pharmaceutical compositions and methods for treating depression, anxiety, and neurodegenerative diseases and cognitive disorders, such as dementia and Alzheimer's disease, by administering same are provided. The compositions comprise dextromethorphan in combination with quinidine.

### BACKGROUND OF THE INVENTION

Dementia is a neurological disease that results in loss of mental capacity and is associated with widespread reduction in the number of nerve cells and brain tissue shrinkage. Memory is the mental capacity most often affected by dementia. The memory loss may first manifest itself in simple absentmindedness, a tendency to forget or misplace things, or to repeat oneself in conversation. As the dementia progresses, the loss of memory broadens in scope until the patient can no longer remember basic social and survival skills and function independently. Dementia can also result in a decline in the patient's language skills, spatial or temporal orientation, judgment, or other cognitive capacities. Dementia tends to run an insidious and progressive course.

Alzheimer's disease is a degenerative brain disorder presented clinically by progressive loss of memory, cognition, reasoning, judgment, and emotional stability that gradually leads to profound mental deterioration and ultimately death. Individuals with Alzheimer's disease exhibit characteristic beta amyloid deposits in the brain (beta amyloid plaques) and in cerebral blood vessels (beta amyloid angiopathy) as well as neurofibrillary tangles. On autopsy of Alzheimer's disease patients, large numbers of these lesions, which are believed to be a causative precursor or factor in the development of disease, are generally found in areas of the human brain important for memory and cognitive function. Smaller numbers are found in the brains of most aged humans not showing clinical symptoms of Alzheimer's disease. Beta amyloid plaques and beta amyloid angiopathy also characterize the brains of individuals with Down's syndrome (Trisomy 21) and Hereditary Cerebral Hemorrhage with Beta amyloidosis of the Dutch-Type, and other such disorders.

Vascular dementia (VaD) is defined as the loss of cognitive function resulting from ischemic, ischemic-hypoxic, or hemorrhagic brain lesions as a result of cardiovascular diseases and cardiovascular pathologic changes. Vascular dementia is a chronic disorder and the symptoms of vascular dementia include cognitive loss, headaches, insomnia and memory loss. Vascular dementia may be caused by multiple strokes (multi-infarct dementia or post-stroke dementia) but also by single strategic strokes, multiple lacunes, and hypoperfusive lesions such as border zone infarcts and ischemic periventricular leukoencephalopathy (Binswanger's disease).

Patients suffering from neurodegenerative diseases, brain damage caused by stroke, dementia, Alzheimer's disease, or head injury often are afflicted with emotional problems associated with the disease or injury. The terms involuntary emotional expression disorder (IEED), emotional lability, and pseudobulbar affect are used by psychiatrists and neurologists to refer to a set of symptoms that are often observed in patients who have suffered a brain insult such as a head injury, stroke, brain tumor, or encephalitis, or who are suffering from a progressive neurodegenerative disease such as Amyotrophic Lateral Sclerosis (ALS, also called motor neuron disease or Lou Gehrig's disease), Parkinson's disease, Alzheimer's disease, or multiple sclerosis (MS). In the great majority of such cases, emotional lability occurs in patients who have bilateral damage (damage which affects both hemispheres of the brain) involving subcortical forebrain structures.

Involuntary emotional expression disorder is distinct from clinical forms of reactive or endogenous depression, and is characterized by intermittent spasmodic outbursts of emotion, such as anger, or expressions of irritability or frustration at inappropriate times or in the absence of any particular provocation. The feelings that accompany emotional lability are often described in words such as “disconnectedness,” since patients are fully aware that an outburst is not appropriate in a particular situation, but they do not have control over their emotional displays.

Emotional lability or pseudobulbar affect becomes a clinical problem when the inability to control emotional outbursts interferes in a substantial way with the ability to engage in family, personal, or business affairs. These symptoms can occur even though the patient still has more than enough energy and stamina to do the physical tasks necessary to interact with other people. Such outbursts, along with the feelings of annoyance, inadequacy, and confusion that they usually generate and the visible effects they have on other people, can severely aggravate the other symptoms of the disease; they lead to feelings of ostracism, alienation, and isolation, and they can render it very difficult for friends and family members to provide tolerant and caring emotional support for the patient.

People with diseases such as Alzheimer's also often have behavior problems in the late afternoon and evening. They may become demanding, suspicious, upset or disoriented, see or hear things that are not there and believe things that aren't true. Or they may pace or wander around the house when others are sleeping. While experts are unsure how or why this behavior occurs, they suspect that the problem of late afternoon confusion, which is sometimes called “sundowning,” or “sundown syndrome,” may be due to these factors: the person with Alzheimer's can't see well in dim light and becomes confused; the impaired person may have a hormone imbalance or a disturbance in his/her “biological clock”; the person with Alzheimer's gets tired at the end of the day and is less able to cope with stress; the person is involved in activities all day long and grows restless if there's nothing to do in the late afternoon or evening; the caregiver communicates fatigue and stress to the person with Alzheimer's and the person becomes anxious.

Recent estimates indicate that more than 19 million Americans over the age of 18 years experience a depressive illness each year. The American Psychiatric Association recognizes several types of clinical depression, including mild depression (dysthymia), major depression, and bipolar disorder (manic-depression). Depression is defined by a constellation of chronic symptoms that include sleep problems, appetite problems, anhedonia or lack of energy, feelings of worthlessness or hopelessness, difficulty concentrating, suicidal thoughts, mood swings (feelings of sadness, abandonment, humiliation, devaluing), psychomotor inhibition (fatigue, daily powerlessness, difficulty in concentration), manifest anxiety (often in the foreground), and quasi-constant somatic difficulties (oppression, spasms, disturbed sleep, loss of appetite, sexual dysfunction). Approximately 9.2 million Americans suffer from major depression, and approximately 15 percent of all people who suffer from major depression take their own lives. Bipolar disorder involves major depressive episodes alternating with high-energy periods of rash behavior, poor judgment, and grand delusions. An estimated one percent of the American population experiences bipolar disorder annually.

The discovery of antidepressants at the end of the fifties marked a veritable therapeutic revolution in the world of neuropsychiatry. Tricyclic antidepressants (TCA) with amitriptyline and imipramine were the first to be discovered, followed by inhibitors of monoamine oxydase (IMAO), irreversible and non-selective, such as phenelzine (hydrazine), pargyline (class of acetylenics) and iproniazude (Marsilid). Undesirable effects, in particular orthostatic hypotension, dryness in the mouth, drowsiness, constipation, adaptation disorders, but also a proconvulsivant effect and cardiotoxicity of TCA (especially in the event of overdose) and hypertensive crises of inhibitors of monoamine oxydase (interactions with alimentary tyramine, as well as numerous medicinal interactions) have shunted research towards novel molecules of identical therapeutic efficacy, but having better acceptability.

Selective serotonin reuptake inhibitors (SSRIs) have become first choice therapeutics in the treatment of depression, certain forms of anxiety and social phobias, because they are effective, well tolerated and have a favorable safety profile compared to the classic tricyclic antidepressants. Since the introduction of elective serotonin reuptake inhibitors, many patients have been effectively treated with anti-depressant medication. However, clinical studies on depression and anxiety disorders indicate that non-response to elective serotonin reuptake inhibitors is substantial, up to 30%. Another, often neglected, factor in antidepressant treatment is compliance, which has a rather profound effect on the patient's motivation to continue pharmacotherapy. First of all, there is the delay in therapeutic effect of elective serotonin reuptake inhibitors. Sometimes symptoms even worsen during the first weeks of treatment. Secondly, sexual dysfunction is a side effect common to all elective serotonin reuptake inhibitors. The serotoninergic syndrome, often misunderstood, is associated with certain overdoses or interactions and justifies an immediate halt to treatment. It can cause hospitalization, and in exceptional circumstances the involvement of vital prognosis. It links a set of symptoms of digestive order (diarrhea), vegetative: (sweating, thermal deregulation, hypo- or hypertension), motor (myoclonia, trembling), neuropsychic (confusion, agitation, even coma). New medications to treat depression are introduced almost every year, and research in this area is ongoing. However, an estimated 10 to 30 percent of depressed patients taking an anti-depressant are partially or totally resistant to the treatment. Those who suffer from treatment-resistant depression have almost no alternatives.

Anxiety is an emotional condition characterized by feelings such as apprehension and fear accompanied by physical symptoms such as tachycardia, increased respiration, sweating and tremor. It is a normal emotion but when it is severe and disabling it becomes pathological. Anxiety disorders are generally treated using benzodiazepine sedative/anti-anxiety agents. Potent benzodiazepines are effective in panic disorder as well as in generalized anxiety disorder, however, the risks associated with the drug dependency may limit their long-term use, 5-H1A receptor partial agonists also have useful anxiolytic and other pyschotropic activity, and less likelihood of sedation and dependence.

### SUMMARY OF THE INVENTION

There is an urgent need exists for pharmaceutical agents capable of treating symptoms associated with dementia or Alzheimer's disease. There also remains a need for additional or improved forms of treatment for involuntary emotional expression disorder (including inappropriate expression of anger, irritability, and frustration), sundown syndrome, and other disorders, such as chronic pain. Such a treatment preferably provides at least some degree of improvement compared to other known drugs, in at least some patients. A method for treating emotional lability in at least some patients suffering from neurological impairment, such as a progressive neurological disease, is desirable.

Moreover, in view of the short-comings of existing antidepressant and anti-anxiety therapy, there is a need for new, safe and effective treatments for depression and anxiety. There is a need to develop alternative treatments for those patients who suffer from treatment-resistant depression or anxiety. There is also a need for treatments for depression and anxiety which lack, or have minimal, undesirable side effects, e.g., such as are observed in tricyclic antidepressants, SSRIs, and benzodiazepines.

Methods of treatment of depression and/or anxiety that can provide one or more of these benefits involve administering dextromethorphan in combination with a dosage of quinidine. The methods and compositions of the preferred embodiments are also useful for treating social anxiety disorder, posttraumatic stress disorder (PTSD), panic disorder, eating disorders (anorexia, bulimia), obsessive-compulsive disorder (OCD), and premenstrual dysphoric disorder (PMDD).

In a first aspect, a method for treating depression is provided, the method comprising administering to a patient in need thereof dextromethorphan in combination with quinidine, wherein an amount of dextromethorphan administered comprises from about 20 mg/day to about 200 mg/day, and wherein an amount of quinidine administered comprises from about 10 mg/day to less than about 50 mg/day.

In an embodiment of the first aspect, the amount of quinidine administered comprises from about 20 mg/day to about 45 mg/day.

In an embodiment of the first aspect, the amount of dextromethorphan administered comprises from about 20 mg/day to about 60 mg/day.

In an embodiment of the first aspect, at least one of the quinidine and the dextromethorphan is in a form of a pharmaceutically acceptable salt.

In an embodiment of the first aspect, at least one of the quinidine and the dextromethorphan is in a form of a pharmaceutically acceptable salt selected from the group consisting of salts of alkali metals, salts of lithium, salts of sodium, salts of potassium, salts of alkaline earth metals, salts of calcium, salts of magnesium, salts of lysine, salts of N,N′-dibenzylethylenediamine, salts of chloroprocaine, salts of choline, salts of diethanolamine, salts of ethylenediamine, salts of meglumine, salts of procaine, salts of tris, salts of free acids, salts of free bases, inorganic salts, salts of sulfate, salts of hydrochloride, and salts of hydrobromide.

In an embodiment of the first aspect, the quinidine comprises quinidine sulfate and the dextromethorphan comprises dextromethorphan hydrobromide, and wherein an amount of quinidine sulfate administered comprises from about 30 mg/day to 60 mg/day and wherein an amount of dextromethorphan hydrobromide administered comprises from about 30 mg/day to about 60 mg/day.

In an embodiment of the first aspect, the dextromethorphan and the quinidine are administered in a combined dose, and wherein a weight ratio of dextromethorphan to quinidine in the combined dose is about 1:1.25 or less

In a second aspect, a method for treating anxiety is provided, the method comprising administering to a patient in need thereof dextromethorphan in combination with quinidine, wherein an amount of dextromethorphan administered comprises from about 20 mg/day to about 200 mg/day, and wherein an amount of quinidine administered comprises from about 10 mg/day to less than about 50 mg/day.

In an embodiment of the second aspect, the amount of quinidine administered comprises from about 20 mg/day to about 45 mg/day.

In an embodiment of the second aspect, the amount of dextromethorphan administered comprises from about 20 mg/day to about 60 mg/day.

In an embodiment of the second aspect, at least one of the quinidine and the dextromethorphan is in a form of a pharmaceutically acceptable salt.

In an embodiment of the second aspect, at least one of the quinidine and the dextromethorphan is in a form of a pharmaceutically acceptable salt selected from the group consisting of salts of alkali metals, salts of lithium, salts of sodium, salts of potassium, salts of alkaline earth metals, salts of calcium, salts of magnesium, salts of lysine, salts of N,N′-dibenzylethylenediamine, salts of chloroprocaine, salts of choline, salts of diethanolamine, salts of ethylenediamine, salts of meglumine, salts of procaine, salts of tris, salts of free acids, salts of free bases, inorganic salts, salts of sulfate, salts of hydrochloride, and salts of hydrobromide.

In an embodiment of the second aspect, the quinidine comprises quinidine sulfate and the dextromethorphan comprises dextromethorphan hydrobromide, and wherein an amount of quinidine sulfate administered comprises from about 30 mg/day to 60 mg/day and wherein an amount of dextromethorphan hydrobromide administered comprises from about 30 mg/day to about 60 mg/day.

In an embodiment of the second aspect, the dextromethorphan and the quinidine are administered in a combined dose, and wherein a weight ratio of dextromethorphan to quinidine in the combined dose is about 1:1.25 or less.

In a third aspect, a method for treating symptoms associated with a neurodegenerative disorder is provided, the method comprising administering to a patient in need thereof dextromethorphan in combination with quinidine, wherein an amount of dextromethorphan administered comprises from about 20 mg/day to about 200 mg/day, and wherein an amount of quinidine administered comprises from about 10 mg/day to less than about 50 mg/day.

In an embodiment of the third aspect, the neurodegenerative disorder is Alzheimer's disease.

In an embodiment of the third aspect, the neurodegenerative disorder is dementia.

In an embodiment of the third aspect, the neurodegenerative disorder is multiple sclerosis.

In an embodiment of the third aspect, the neurodegenerative disorder is amyotrophic lateral sclerosis.

In an embodiment of the third aspect, the neurodegenerative disorder is Parkinson's disease.

In an embodiment of the third aspect, the neurodegenerative disorder is Huntington's disease.

In an embodiment of the third aspect, the amount of quinidine administered comprises from about 20 mg/day to about 45 mg/day.

In an embodiment of the third aspect, the amount of dextromethorphan administered comprises from about 20 mg/day to about 60 mg/day.

In an embodiment of the third aspect, at least one of the quinidine and the dextromethorphan is in a form of a pharmaceutically acceptable salt.

In an embodiment of the third aspect, at least one of the quinidine and the dextromethorphan is in a form of a pharmaceutically acceptable salt selected from the group consisting of salts of alkali metals, salts of lithium, salts of sodium, salts of potassium, salts of alkaline earth metals, salts of calcium, salts of magnesium, salts of lysine, salts of N,N′-dibenzylethylenediamine, salts of chloroprocaine, salts of choline, salts of diethanolamine, salts of ethylenediamine, salts of meglumine, salts of procaine, salts of tris, salts of free acids, salts of free bases, inorganic salts, salts of sulfate, salts of hydrochloride, and salts of hydrobromide.

In an embodiment of the third aspect, the quinidine comprises quinidine sulfate and the dextromethorphan comprises dextromethorphan hydrobromide, and wherein an amount of quinidine sulfate administered comprises from about 30 mg/day to 60 mg/day and wherein an amount of dextromethorphan hydrobromide administered comprises from about 30 mg/day to about 60 mg/day.

In an embodiment of the third aspect, the dextromethorphan and the quinidine are administered in a combined dose, and wherein a weight ratio of dextromethorphan to quinidine in the combined dose is about 1:1.25 or less.

### BRIEF DESCRIPTION OF THE DRAWINGS

FIG. 1 illustrates the principal mechanisms by which dextromethorphan is proposed to exert its neuroprotective effects at the cellular level.

FIG. 2 is the treatment schedule of the Emotional Lability Clinical Study.

FIG. 3 depicts the patient distribution of the Emotional Lability Clinical Study.

### DETAILED DESCRIPTION OF THE PREFERRED EMBODIMENT

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

Emerging evidence suggests that the amino acid neurotransmitter systems are associated with the pathophysiology and treatment of mood disorders (Sanacora et al., Ann N Y Acad Sci. 2003 November; 1003:292-308). In particular, glutamate and gamma-amino butyric acid (GABA) systems are emerging as targets for development of medications for mood disorders. There is increasing preclinical and clinical evidence that antidepressant drugs directly or indirectly reduce N-methyl-D-aspartate glutamate receptor function. Drugs that reduce glutamatergic activity or glutamate receptor-related signal transduction may also have antimanic effects. Recent studies employing magnetic resonance spectroscopy also suggest that unipolar, but not bipolar, depression is associated with reductions in cortical GABA levels. Antidepressant and mood-stabilizing treatments also appear to raise cortical GABA levels and to ameliorate GABA deficits in patients with mood disorders. The preponderance of available evidence suggests that glutamatergic and GABAergic modulation may be an important property of available antidepressant and mood-stabilizing agents (Krystal et al., Mol Psychiatry. 2002; 7 Suppl 1:S71-80).

The monoamine theory has implicated abnormalities in serotonin and norepinephrine in the pathophysiology of major depression and bipolar illness and contributed greatly to our understanding of mood disorders and their treatment. Nevertheless, some limitations of this model still exist that require researchers and clinicians to seek further explanation and develop novel interventions that reach beyond the confines of the monoaminergic systems. Recent studies have provided strong evidence that glutamate and other amino acid neurotransmitters are involved in the pathophysiology and treatment of mood disorders. Studies employing in vivo magnetic resonance spectroscopy have revealed altered cortical glutamate levels in depressed subjects. Consistent with a model of excessive glutamate-induced excitation in mood disorders, several antiglutamatergic agents, such as riluzole and lamotrigine, have demonstrated potential antidepressant efficacy. Glial cell abnormalities commonly associated with mood disorders may at least partly account for the impairment in glutamate action since glial cells play a primary role in synaptic glutamate removal. A hypothetical model of altered glutamatergic function in mood disorders is proposed in conjunction with potential antidepressant mechanisms of antiglutamatergic agents. Further studies elucidating the role of the glutamatergic system in the pathophysiology of mood and anxiety disorders and studies exploring the efficacy and mechanism of action of antiglutamatergic agents in these disorders, are likely to provide new targets for the development of novel antidepressant agents (Kugaya et al., CNS Spectr. 2005 October; 10(10):808-19).

Most patients with obsessive-compulsive disorder (OCD) show only partial reduction of symptoms with standard therapy. Recent imaging data suggests glutamatergic dysfunction in the corticostriatal pathway in OCD (Coric et al., Biol Psychiatry. 2005 Sep. 1; 58(5):424-8).

Advances made in diverse areas of neuroscience suggest that neurotransmitter systems, additional to the monoaminergic, contribute to the pathophysiology of mood disorders. This ever accruing body of preclinical and clinical research is providing increased recognition of the contribution made by amino acid neurotransmitters to the neurobiology of mood disorders (Kendell et al., Expert Opin Ther Targets. 2005 February; 9(1):153-68).

Methods of treating mental disorders, including anxiety disorders such as obsessive-compulsive disorder, are provided. The methods comprise administering an effective amount of a glutamate modulator, e.g., dextromethorphan, to an individual in need thereof are described in PCT International Publication No. WO 06/108055-A1 to Coric et al.

Because of the possibility that a process involving glutamate is etiologically implicated in depression, anxiety, and related mood disorders, administration of dextromethorphan (DM) can be an effective treatment. Dextromethorphan is a noncompetitive antagonist of the N-methyl-D-aspartate-sensitive ionotropic glutamate receptor, and it acts by reducing the level of excitatory activity. However, dextromethorphan is extensively metabolized to dextrorphan (DX) and a number of other metabolites. Cytochrome P450 2D6 (CYP2D6) is the key enzyme responsible for the formation of dextrorphan from dextromethorphan. A subset of the population, 5 to 10% of Caucasians, has reduced activity of this enzyme (Hildebrand et al., Eur. J. Clin. Pharmacol., 1989; 36:315-318). Such individuals are referred to as “poor metabolizers” of dextromethorphan in contrast to the majority of individuals who are referred to as “extensive metabolizers” of dextromethorphan (Vetticaden et al., Pharm. Res., 1989; 6:13-9).

A number of in vitro studies have been undertaken to determine the types of drugs that inhibit CYP2D6 activity. Quinidine (Q) is one of the most potent of those that have been studied (Inaba et al., Br. J. Clin. Pharmacol., 1986; 22:199-200). These observations led to the hypothesis that concomitant dosing with quinidine could increase the concentration of dextromethorphan in plasma.

A number of chronic disorders other than emotional lability also have symptoms which are known to be very difficult to treat, and often fail to respond to safe, non-addictive, and non-steroid medications. Disorders such as intractable coughing fail to respond to conventional medicines and are typically treated by such drugs as codeine, morphine, or the anti-inflammatory steroid prednisone. These drugs are unacceptable for long-term treatment due to dangerous side effects, long-term risks to the patient's health, or the danger of addiction. There has been no satisfactory treatment for the severe itching and rash associated with dermatitis. Drugs such as prednisone and even tricyclic antidepressants, as well as topical applications have been employed, but do not appear to offer substantial and consistent relief. Chronic pain due to conditions such as stroke, cancer, and trauma, as well as neuropathic pain resulting from conditions such as diabetes and shingles (herpes zoster), for example, is also a problem which resists treatment. Neuropathic pain includes, for example, diabetic neuropathy, postherpetic neuralgia, phantom limb pain, trigeminal neuralgia, and sciatica. Postherpetic neuralgia (PHN) is a complication of shingles and occurs in approximately ten percent of patients with herpes zoster. The incidence of postherpetic neuralgia increases with age. Diabetic neuropathy is a common complication of diabetes which increases with the duration of the disease. The pain for these types of neuropathies has been described as a burning steady pain often punctuated with stabbing pains, pins and needles pain, and toothache-like pain. The skin can be sensitive with dysesthetic sensations to even light touch and clothing. The pain can be exacerbated by activity, temperature change, and emotional upset. The pain can be so severe as to preclude daily activities or result in sleep disturbance or anorexia. The mechanisms involved in producing pain of these types are not well understood, but may involve degeneration of myelinated nerve fibers. It is known that in diabetic neuropathy, both small and large nerve fibers deteriorate resulting in reduced thresholds for tolerance of thermal sensitivity, pain, and vibration. Dysfunction of both large and small fiber functions is more severe in the lower limbs when pain develops. Most of the physiological measurements of nerves that can be routinely done in patients experiencing neuropathic pain demonstrate a slowing of nerve conduction over time. To date, treatment for neuropathic pain has been less than universally successful. Chronic pain is estimated to affect millions of people.

The chemistry of dextromethorphan and its analogs is described in various references such as Rodd, E. H., Ed., Chemistry of Carbon Compounds, Elsevier Publ., N.Y., 1960; Goodman and Gilman's Pharmacological Basis of Therapeutics; Choi, Brain Res., 1987, 403: 333-336; and U.S. Pat. No. 4,806,543. Its chemical structure is as follows:

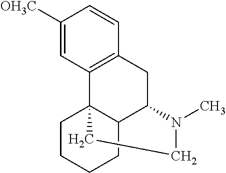

Dextromethorphan is the common name for (+)-3-methoxy-N-methylmorphinan. It is one of a class of molecules that are dextrorotatory analogs of morphine-like opioids. The term “opiate” refers to drugs that are derived from opium, such as morphine and codeine. The term “opioid” is broader. It includes opiates, as well as other drugs, natural or synthetic, which act as analgesics and sedatives in mammals.

Most of the addictive analgesic opiates, such as morphine, codeine, and heroin, are levorotatory stereoisomers (they rotate polarized light in the so-called left-handed direction). They have four molecular rings in a configuration known as a “morphinan” structure, which is depicted as follows:

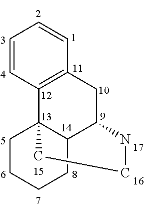 [SMILES: c1ccc2c(c1)C[C@@H]1NCC[C@]23CCCCC13]

In this depiction, the carbon atoms are conventionally numbered as shown, and the wedge-shaped bonds coupled to carbon atoms 9 and 13 indicate that those bonds rise out of the plane of the three other rings in the morphinan structure. Many analogs of this basic structure (including morphine) are pentacyclic compounds that have an additional ring formed by a bridging atom (such as oxygen) between the number 4 and 5 carbon atoms.

Many dextrorotatory analogs of morphine are much less addictive than the levorotatory compounds. Some of these dextrorotatory analogs, including dextromethorphan and dextrorphan, are enantiomers of the morphinan structure. In these enantiomers, the ring that extends out from carbon atoms 9 and 13 is oriented in the opposite direction from that depicted in the above structure.

While not wishing to be limited to any particular mechanism of action, dextromethorphan is known to have at least three distinct receptor activities which affect central nervous system neurons. First, it acts as an antagonist at N-methyl-D-aspartate (NMDA) receptors. NMDA receptors are one of three major types of excitatory amino acid (EAA) receptors in central nervous system neurons. Since activation of NMDA receptors causes neurons to release excitatory neurotransmitter molecules (primarily glutamate, an amino acid), the blocking activity of dextromethorphan at these receptors reduces the level of excitatory activity in neurons having these receptors. Dextromethorphan is believed to act at the phencyclidine (PCP) binding site, which is part of the NMDA receptor complex. Dextromethorphan is relatively weak in its NMDA antagonist activity, particularly compared to drugs such as MK-801 (dizocilpine) and phencyclidine. Accordingly, when administered at approved dosages, dextromethorphan is not believed to cause the toxic side effects (discussed in U.S. Pat. No. 5,034,400 to Olney) that are caused by powerful NMDA antagonists such as MK-801 or PCP.

Dextromethorphan also functions as an agonist at certain types of inhibitory receptors; unlike EAA receptors, activation of inhibitory receptors suppresses the release of excitatory neurotransmitters by affected cells. Initially, these inhibitory receptors were called sigma opiate receptors. However, questions have been raised as to whether they are actually opiate receptors, so they are now generally referred to as sigma (a) receptors. Subsequent experiments showed that dextromethorphan also binds to another class of inhibitory receptors that are closely related to, but distinct from, sigma receptors. The evidence, which indicates that non-sigma inhibitory receptors exist and are bound by dextromethorphan, is that certain molecules which bind to sigma receptors are not able to completely block the binding of dextromethorphan to certain types of neurons that are known to have inhibitory receptors (Musacchio et al., Cell Mol. Neurobiol., 1988 June, 8(2):149-56; Musacchio et al., J. Pharmacol. Exp. Ther., 1988 November, 247(2):424-31; Craviso et al., Mol. Pharmacol., 1983 May, 23(3):629-40; Craviso et al., Mol. Pharmacol., 1983 May, 23(3):619-28; and Klein et al., Neurosci. Lett., 1989 Feb. 13, 97(1-2):175-80). These receptors are generally called “high-affinity dextromethorphan receptors” or simply “dextromethorphan receptors” in the scientific literature. As used herein, the phrase “dextromethorphan-binding inhibitory receptors” includes both sigma and non-sigma receptors which undergo affinity-binding reactions with dextromethorphan and which, when activated by dextromethorphan, suppress the release of excitatory neurotransmitters by the affected cells (Largent et al., Mol. Pharmacol., 1987 December, 32(6):772-84).

Dextromethorphan also decreases the uptake of calcium ions (Ca′) by neurons. Calcium uptake, which occurs during transmission of nerve impulses, involves at least two different types of channels, known as N-channels and L-channels. Dextromethorphan suppressed calcium uptake fairly strongly in certain types of cultured neurons (synaptosomes) which contain N-channels; it also suppressed calcium uptake, although less strongly, in other cultured neurons (PC12 cells) which contain L-channels (Carpenter et al., Brain Res., 1988 Jan. 26, 439(1-2):372-5).

An increasing body of evidence indicates dextromethorphan has therapeutic potential for treating several neuronal disorders (Zhang et al., Clin. Pharmacol. Ther. 1992; 51: 647-655; Palmer G C, Curr. Drug Targets, 2001; 2: 241-271; and Liu et al., J. Pharmacol. Exp. Ther. 2003; 21: 21; Kim et al., Life Sci., 2003; 72: 769-783). Pharmacological studies demonstrate that dextromethorphan is a noncompetitive NMDA antagonist that has neuroprotective, anticonvulsant and antinociceptive activities in a number of experimental models (Desmeules et al., J. Pharmacol. Exp. Ther., 1999; 288: 607-612). In addition to acting as an NMDA antagonist, both dextromethorphan and its primary metabolite, dextrorphan, bind to sigma-1 sites, inhibit calcium flux channels and interact with high voltage-gated sodium channels (Dickenson et al., Neuropharmacology, 1987; 26: 1235-1238; Carpenter et al., Brain Res., 1988; 439: 372-375; Netzer et al., Eur. J. Pharmacol., 1993; 238: 209-216). Recent reports indicate that an additional neuroprotective mechanism of dextromethorphan may include interference with the inflammatory responses associated with some neurodegenerative disorders that include Parkinson's disease and Alzheimer's disease (Liu et al., J. Pharmacol. Exp. Ther., 2003; 21: 21). The potential efficacy of dextromethorphan as a neuroprotectant was explored in limited clinical trials in patients with amyotrophic lateral sclerosis (Gredal et al., Neurol. Acta Neurol. Scand. 1997; 96: 8-13; Blin et al., Clin. Neuropharmacol., 1996; 19: 189-192) Huntington's disease (Walker et al., Clin. Neuropharmacol., 1989; 12: 322-330) and Parkinson's disease (Chase et al., Neurol. J. Neurol., 2000; 247 Suppl 2: 1136-42). Dextromethorphan was also examined in patients with various types of neuropathic pain (Mcquay et al., Pain, 1994; 59: 127-133; Vinik A I, Am. J. Med., 1999; 107: 17S-26S; Weinbroum et al., Can. J. Anaesth., 2000; 47: 585-596; Sang et al., Anesthesiology, 2002; 96: 1053-1061; Heiskanen et al., Pain, 2002; 96: 261-267; Ben Abraham et al., Clin. J. Pain, 2002; 18: 282-285; Sang C N, J. Pain Symptom Manage., 2000; 19: S21-25). Although the pharmacological profile of dextromethorphan points to clinical efficacy, most clinical trials have been disappointing with equivocal efficacy for dextromethorphan compared to placebo treatment.

Several investigators suggested that the limited benefit seen with dextromethorphan in clinical trials is associated with rapid hepatic metabolism that limits systemic drug concentrations. In one trial in patients with Huntington's disease, plasma concentrations were undetectable in some patients after dextromethorphan doses that were eight times the maximum antitussive dose (Walker et al., Clin. Neuropharmacol., 1989; 12: 322-330).

As discussed above, dextromethorphan undergoes extensive hepatic 0-demethylation to dextrorphan that is catalyzed by CYP2D6. This is the same enzyme that is responsible for polymorphic debrisoquine hydroxylation in humans (Schmid et al., Clin. Pharmacol. Ther., 1985; 38: 618-624). An alternate pathway is mediated primarily by CYP3A4 and N-demethylation to form 3-methoxymorphinan (Von Moltke et al., J. Pharm. Pharmacol., 1998; 50: 997-1004). Both dextrorphan and 3-methoxymorphinan can be further demethylated to 3-hydroxymorphinan that is then subject to glucuronidation. The metabolic pathway that converts dextromethorphan to dextrorphan is dominant in the majority of the population and is the principle for using dextromethorphan as a probe to phenotype individuals as CYP2D6 extensive and poor metabolizers (Kupfer et al., Lancet 1984; 2: 517-518; Guttendorf et al., Ther. Drug Monit., 1988; 10: 490-498). Approximately 7% of the Caucasian population shows the poor metabolizer phenotype, while the incidence of poor metabolizer phenotype in Chinese and Black African populations is lower (Droll et al., Pharmacogenetics, 1998; 8: 325-333). A study examining the ability of dextromethorphan to increase pain threshold in extensive and poor metabolizers found antinociceptive effects of dextromethorphan were significant in poor metabolizers but not in extensive metabolizers (Desmeules et al., J. Pharmacol. Exp. Ther., 1999; 288: 607-612). The results are consistent with direct effects of parent dextromethorphan rather than the dextrorphan metabolite on neuromodulation.

One approach for increasing systemically available dextromethorphan is to coadminister the CYP2D6 inhibitor, quinidine, to protect dextromethorphan from metabolism (Zhang et al., Clin. Pharmacol. Ther. 1992; 51: 647-655). Quinidine administration can convert subjects with extensive metabolizer phenotype to poor metabolizer phenotype (Inaba et al., Br. J. Clin. Pharmacol., 1986; 22: 199-200). When this combination therapy was tried in amyotrophic lateral sclerosis patients it appeared to exert a palliative effect on symptoms of pseudobulbar affect (Smith et al., Neurol., 1995; 54: 604P). Combination treatment with dextromethorphan and quinidine also appeared effective for patients with chronic pain that could not be adequately controlled with other medications. This observation is consistent with a report that showed dextromethorphan was effective in increasing pain threshold in poor metabolizers and in extensive metabolizers given quinidine, but not in extensive metabolizers (Desmeules et al., J. Pharmacol. Exp. Ther., 1999; 288: 607-612). To date, most studies have used quinidine doses ranging from 50 to 200 mg to inhibit CYP2D6 mediated drug metabolism, but no studies have identified a minimal dose of quinidine for enzyme inhibition.

The highly complex interactions between different types of neurons having varying populations of different receptors, and the cross-affinity of different receptor types for dextromethorphan as well as other types of molecules which can interact with some or all of those same types of receptors, render it very difficult to attribute the overall effects of dextromethorphan to binding activity at any particular receptor type. Nevertheless, it is believed that dextromethorphan suppresses neuronal activity by means of at least three molecular functions: it reduces activity at (excitatory) NMDA receptors; it inhibits neuronal activity by binding to certain types of inhibitory receptors; and it suppresses calcium uptake through N-channels and L-channels.

Unlike some analogs of morphine, dextromethorphan has little or no agonist or antagonist activity at various other opiate receptors, including the mu (μ) and kappa (κ) classes of opiate receptors. This is highly desirable, since agonist or antagonist activity at those opiate receptors can cause undesired side effects such as respiratory depression (which interferes with breathing) and blockade of analgesia (which reduces the effectiveness of pain-killers).

Accordingly, cognitive or neurodegenerative disorders such as dementia or Alzheimer's disease, or anger, frustration, or irritability associated with involuntary emotional expression disorder, as well as depression, and anxiety can be treated in at least some patients by means of administering a drug which functions as an antagonist at NMDA receptors and as an agonist at dextromethorphan-binding inhibitory receptors, and wherein the drug is also characterized by a lack of agonist or antagonist activity at mu or kappa opiate receptors, namely, dextromethorphan.

### Metabolism of Dextromethorphan

It has long been known that in most people (estimated to include about 90% of the general population in the United States), dextromethorphan is rapidly metabolized and eliminated by the body (Ramachander et al., J. Pharm. Sci., 1977 July, 66(7):1047-8; and Vetticaden et al., Pharm. Res., 1989 January, 6(1):13-9). This elimination is largely due to an enzyme known as the P450 2D6 (or IID6) enzyme, which is one member of a class of oxidative enzymes that exist in high concentrations in the liver, known as cytochrome P450 enzymes (Kronbach et al., Anal. Biochem., 1987 April, 162(1):24-32; and Dayer et al., Clin. Pharmacol. Ther., 1989 January, 45(1):34-40). In addition to metabolizing dextromethorphan, the P450 2D6 isozyme also oxidizes sparteine and debrisoquine. It is known that the P450 2D6 enzyme can be inhibited by a number of drugs, particularly quinidine (Brinn et al., Br. J. Clin. Pharmacol., 1986 August, 22(2):194-7; Inaba et al., Br. J. Clin. Pharmacol., 1986 August, 22(2):199-200; Brosen et al., Pharmacol. Toxicol., 1987 April, 60(4):312-4; Otton et al., Drug Metab. Dispos., 1988 January-February, 16(1):15-7; Otton et al., J. Pharmacol. Exp. Ther., 1988 October, 247(1):242-7; Funck-Brentano et al., Br. J. Clin. Pharmacol., 1989 April, 27(4):435-44; Funck-Brentano et al., J. Pharmacol. Exp. Ther., 1989 April, 249(1):134-42; Nielsen et al., Br. J. Clin. Pharmacol., 1990 March, 29(3):299-304; Broly et al., Br. J. Clin. Pharmacol., 1989 July, 28(1):29-36).

Patients who lack the normal levels of P450 2D6 activity are classified in the medical literature as “poor metabolizers,” and doctors are generally warned to be cautious about administering various drugs to such patients. “The diminished oxidative biotransformation of these compounds in the poor metabolizer (PM) population can lead to excessive drug accumulation, increased peak drug levels, or in some cases, decreased generation of active metabolites . . . Patients with the PM phenotype are at increased risk of potentially serious untoward effects . . . ” (Guttendorf et al., Ther. Drug Monit., 1988, 10(4):490-8, page 490). Accordingly, doctors are cautious about administering quinidine to patients, and rather than using drugs such as quinidine to inhibit the rapid elimination of dextromethorphan, researchers working in this field have administered very large quantities (such as 750 mg/day) of dextromethorphan to their patients, even though this is known to introduce various problems (Walker et al., Clin Neuropharmacol., 1989 August, 12(4):322-30; and Albers et al., Stroke, 1991 August, 22(8):1075-7).

DM metabolism is primarily mediated by CYP2D6 in extensive metabolizers. This can be circumvented by co-administration of quinidine, a selective CYP2D6 inhibitor, at quinidine doses 1 to 1.5 logs below those employed for the treatment of cardiac arrhythmias (Schadel et al., J. Clin. Psychopharmacol., 1995; 15:263-9). Blood levels of dextromethorphan increase linearly with dextromethorphan dose following co-administration with quinidine but are undetectable in most subjects given dextromethorphan alone, even at high doses (Zhang et al., Clin. Pharmac. & Therap., 1992; 51:647-55). The observed plasma levels in these individuals thus mimic the plasma levels observed in individuals expressing the minority phenotype where polymorphisms in the gene result in reduced levels of P450 2D6 (poor metabolizers). Unexpectedly, during a study of dextromethorphan and quinidine in amyotrophic lateral sclerosis patients, patients reported that their emotional lability improved during treatment. Subsequently, in a placebo controlled crossover study (N=12) conducted to investigate this, the concomitant administration of dextromethorphan and quinidine administered to amyotrophic lateral sclerosis patients was found to suppress emotional lability (P<0.001 compared to placebo) (Smith et al., Neurology, 1995; 45:A330).

Rapid dextromethorphan elimination may be overcome by co-administration of quinidine along with dextromethorphan (U.S. Pat. No. 5,206,248 to Smith). The chemical structure of quinidine is as follows:

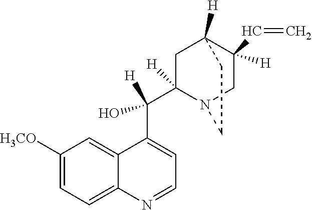

Quinidine co-administration has at least two distinct beneficial effects. First, it greatly increases the quantity of dextromethorphan circulating in the blood. In addition, it also yields more consistent and predictable dextromethorphan concentrations. Research involving dextromethorphan or co-administration of quinidine and dextromethorphan, and the effects of quinidine on blood plasma concentrations, are described in the patent literature (U.S. Pat. Nos. 5,166,207, 5,863,927, 5,366,980, 5,206,248, and U.S. Pat. No. 5,350,756 to Smith). While quinidine is generally preferred for coadministration, other antioxidants, such as those described in Inaba et al., Drug Metabolism and Disposition 13:443-447 (1985), Fonne-Pfister et al., Biochem. Pharmacol. 37:3829-3835 (1988) and Broly et al., Biochem. Pharmacol. 39:1045-1053 (1990), can also be administered. As reported in Inaba et al., agents with a Kᵢ value (Michaelis-Menton inhibition values) of 50 micromolar or lower include nortriptyline, chlorpromazine, domperidone, haloperidol, pipamperone, labetalol, metaprolol, oxprenolol, propranolol, timolol, mexiletine, quinine, diphenhydramine, ajmaline, lobeline, papaverine, and yohimbine. Preferred compounds having particularly potent inhibitory activities include yohimbine, haloperidol, ajmaline, lobeline, and pipamperone, which have Kᵢ values ranging from 4 to 0.33 μM. In addition to the antioxidants reported above, it has also been found that fluoxetine, sold by Eli Lilly and Co. under the trade name Prozac, is effective in increasing dextromethorphan concentrations in the blood of some people. Dosages of other antioxidants will vary with the antioxidant, and are determined on an individual basis.

### Neuroprotective Uses of Dextromethorphan

Mounting preclinical evidence has proven that dextromethorphan has important neuroprotective properties in various in vitro and in vivo central nervous system injury models, including focal and global ischemia, seizure, and traumatic brain injury paradigms. Many of these protective actions appear functionally related to its inhibitory effects on glutamate-induced neurotoxicity via NMDA receptor antagonist, sigma-1 receptor agonist, and voltage-gated calcium channel antagonist actions. Dextromethorphan's protection of dopamine neurons in Parkinsonian models may be due to inhibition of neurodegenerative inflammatory responses. Clinical findings indicate that dextromethorphan protects against neuronal damage, when adequate dextromethorphan brain concentrations are attained. Studies have shown promise for treatment of perioperative brain injury, amyotrophic lateral sclerosis, and symptoms of methotrexate neurotoxicity. Dextromethorphan safety/tolerability trials in stroke, neurosurgery, and amyotrophic lateral sclerosis patients demonstrated a favorable safety profile. The compelling preclinical evidence for neuroprotective properties of dextromethorphan, initial clinical neuroprotective findings, and clinical demonstrations that the dextromethorphan/quinidine combination is well tolerated indicate that dextromethorphan/quinidine can be used for the treatment of various acute and degenerative neurological disorders.

As discussed above, dextromethorphan is a non-opioid morphinan derivative that has been used extensively and safely as a nonprescription antitussive for about 50 years. Dextromethorphan is widely used as a cough syrup, and it has been shown to be sufficiently safe in humans to allow its use as an over-the-counter medicine. It is well tolerated in oral dosage form, either alone or with quinidine, at up to 120 milligrams (mg) per day, and a beneficial effect may be observed when receiving a substantially smaller dose (e.g., 30 mg/day) (U.S. Pat. No. 5,206,248 to Smith). Dextromethorphan has a surprisingly complex central nervous system pharmacology and related neuroactive properties that began to be elucidated and to attract the interest of neurologists in the 1980s (Tortella et al. Trends Pharmacol Sci. 1989a; 10:501-7). It is now established that dextromethorphan acts as a low-affinity uncompetitive NMDA receptor antagonist (Tortella et al. Trends Pharmacol Sci. 1989a; 10:501-7; Chou et al. Brain Res. 1999; 821:516-9; Netzer et al. Eur J Pharmacol. 1993; 238:209-16; and Jaffe et al. Neurosci Lett. 1989; 105:227-32), a high affinity sigma-1 receptor agonist (Zhou et al. Eur J Pharmacol. 1991; 206:261-269; and Maurice et al. Brain Res Brain Res Rev. 2001; 37:116-32), and a voltage-gated calcium channel antagonist (Carpenter et al. Brain Res. 1988; 439:372-5; and Church et al. Neurosci Lett. 1991; 124:232-4).

DM has also been shown to decrease potassium-stimulated glutamate release (Annels et al. Brain Res. 1991; 564:341-343), possibly via a sigma receptor-related mechanism (Maurice et al. Prog Neuropsychopharmacol Biol Psychiatry. 1997; 21:69-102). Sigma-1 receptor agonists modulate extracellular calcium influx, as well as intracellular calcium mobilization (Maurice et al. Brain Res Brain Res Rev. 2001; 37:116-32). Other activities of dextromethorphan appear to include weak serotonin reuptake inhibition (Henderson et al. Brain Res. 1992; 594:323-326; and Gillman. Br J Anaesth. 2005; 95:434-41) through proposed high affinity binding to the serotonin transporter (Meoni et al. Br J Pharmacol. 1997; 120:1255-1262)

In vivo, dextromethorphan is quickly 0-demethylated to its primary metabolite, dextrorphan (Pope et al. J Clin Pharmacol. 2004; 44:1132-1142) which has a similar but not identical pharmacological profile, acting at many, but not all, of the same sites, and with different affinities or potencies (Chou et al. Brain Res. 1999; 821:516-9; Jaffe et al. Neurosci Lett. 1989; 105:227-32; Carpenter et al. Brain Res. 1988; 439:372-5; Meoni et al. Br J Pharmacol. 1997; 120:1255-1262; Trube et al. Epilepsia. 1994; 35 Suppl 5:S62-7; Franklin et al. Mol Pharmacol. 1992; 41:134-146; and Walker et al. Pharmacol Rev. 1990; 42:355-402). Several of the pleiotropic effects of dextromethorphan serve to inhibit excitatory responses to glutamate particularly via NMDA receptors, and to block multiple major routes of calcium entry into neurons (Carpenter et al. Brain Res. 1988; 439:372-5; and Church et al. Neurosci Lett. 1991; 124:232-4). Given the unifying excitotoxic hypothesis of neuronal degeneration and death, dextromethorphan's NMDA receptor antagonist, calcium channel antagonist, and possibly sigma-1 receptor agonist properties point toward potential efficacy as a neuroprotective agent.

Abnormally elevated concentrations of glutamate are hypothesized to cause excessive excitation at the NMDA-subtype of glutamate receptors, which leads to excessive influx of sodium chloride and water, causing acute neuronal damage, and calcium, causing delayed and more permanent injury (Collins et al. Ann Intern Med. 1989; 110:992-1000). Considerable evidence supports roles for excitotoxicity in acute disorders such as stroke, epileptic seizures, traumatic brain and spinal cord injury, as well as in chronic, neurodegenerative disorders such as Alzheimer's disease, Parkinson's disease (PD), Huntington's disease (HD), and amyotrophic lateral sclerosis (Mattson. Neuromolecular Med. 2003; 3:65-94). By pharmacologically inhibiting the release and subsequent deleterious actions of glutamate, dextromethorphan can serve to protect neurons in a variety of neurological disease and injury states.

Neuroprotective effects of dextromethorphan were first recognized by Choi, who demonstrated that the drug attenuated glutamate-induced neurotoxicity in neocortical cell cultures (Choi. Brain Res. 1987; 403:333-6). Since this pioneering study, an increasing body of evidence has proved that dextromethorphan possesses significant neuroprotective properties in a variety of preclinical central nervous system injury models (Trube et al. Epilepsia. 1994; 35 Suppl 5:S62-7) dextromethorphan protects against seizure- and ischemia-induced brain damage, hypoxic and hypoglycemic neuronal injury, as well as traumatic brain and spinal cord injury.

Dextromethorphan's protective action in the plethora of in vitro and in vivo experiments is attributed to diverse mechanisms. Dextromethorphan has been shown to possess both anticonvulsant and neuroprotective properties, which appear functionally related to its inhibitory effects on glutamate-induced neurotoxicity (Bokesch et al. Anesthesiology. 1994; 81:470-7). Antagonism of the NMDA receptor/channel complex is implicated as the predominant mechanism (Trube et al. Epilepsia. 1994; 35 Suppl 5:S62-7), but dextromethorphan's action on sigma-1 receptors is also positively correlated with neuroprotective potency (DeCoster et al. Brain Res. 1995; 671:45-53). Notably, dextromethorphan's dual blockade of voltage-gated and receptor-gated calcium channels is proposed to produce a potentially additive or synergistic therapeutic benefit (Jaffe et al. Neurosci Lett. 1989; 105:227-32; and Church et al. Neurosci Lett. 1991; 124:232-4).

Another suggested neuroprotective mechanism of dextromethorphan underlying the antagonism of p-chloroamphetamine (PCA)-induced neurotoxicity is the inhibition of serotonin (5-HT) uptake by this agent (Narita et al. Eur J Pharmacol. 1995; 293:277-80). Finally, it has been recently proposed that dextromethorphan's interference with the inflammatory responses associated with some neurodegenerative disorders such as Parkinson's disease and Alzheimer's disease may be a novel mechanism by which dextromethorphan protects dopamine neurons in Parkinson's disease models (Liu et al. J Pharmacol Exp Ther. 2003; 305:212-8; and Zhang et al. Faseb J. 2004; 18:589-91).

The efficacy of dextromethorphan as a neuroprotectant was also explored in a limited number of small clinical trials in patients with amyotrophic lateral sclerosis and perioperative brain injury. Additional small studies assessed symptom improvement with dextromethorphan in Huntington's disease, Parkinson's disease, and after methotrexate (MTX) neurotoxicity. Dextromethorphan was not found to be neuroprotective in the amyotrophic lateral sclerosis trials, although the doses employed would not be expected to confer neuroprotection (Gredal et al. Acta Neurol Scand. 1997; 96:8-13; Blin et al. Clin Neuropharmacol. 1996; 19:189-192; and Askmark et al. J Neurol Neurosurg Psychiatry. 1993; 56:197-200). In contrast, the study of patients with perioperative brain injury showed significant reductions in EEG sharp wave activity, and reductions in ventricular enlargement and periventricular white matter lesions that did not reach significance in a small sample of patients (Schmitt et al. Neuropediatrics. 1997; 28:191-7). Symptomatic improvement was not found with dextromethorphan in one open-label trial with Huntington's disease patients (Walker et al. Clin Neuropharmacol. 1989; 12:322-30). Dextromethorphan did significantly improve levodopa-associated dyskinesias and off-time (Verhagen et al. Neurology. 1998b; 51:203-206; and Verhagen et al. Mov Disord. 1998c; 13:414-417). Dextromethorphan also ameliorated primary Parkinson's disease signs in two studies (Bonuccelli et al. Lancet. 1992; 340:53; and Saenz et al. Neurology. 1993; 43:15), although a third pilot investigation using lower doses did not corroborate the latter result (Montastruc et al. Mov Disord. 1994; 9:242-243). Notably, dextromethorphan completely resolved neurological deficits associated with MTX neurotoxicity in all of 5 cases, but a larger trial is needed to confirm these preliminary findings (Drachtman et al. Pediatr Hematol Oncol. 2002; 19:319-327).

To date, primarily safety/tolerability studies have been conducted in neurosurgery patients (Steinberg et al. J Neurosurg. 1996; 84:860-6), amyotrophic lateral sclerosis patients (Hollander et al. Ann Neurol. 1994; 36:920-4), patients at risk for brain ischemia (Albers et al. Stroke. 1991; 22:1075-7), or with a history of cerebral ischemia (Albers et al. Clin Neuropharmacol. 1992; 15:509-14). These safety trials demonstrate the feasibility of long-term and high-dose administration of dextromethorphan to patients with conditions associated with glutamate excitotoxicity, although dextromethorphan was associated with dose-related adverse events (Walker et al. Clin Neuropharmacol. 1989; 12:322-30; and Hollander et al. Ann Neurol. 1994; 36:920-4).

Given the favorable safety profile of dextromethorphan and possible preliminary indications of neuroprotective potential in perioperative brain injury (Albers et al. Stroke. 1991; 22:1075-7; and Albers et al. Clin Neuropharmacol. 1992; 15:509-14), further studies are warranted. Several investigators suggested that the limited benefit seen with dextromethorphan in clinical trials is associated with the rapid hepatic metabolism of dextromethorphan to dextrorphan, which limits systemic drug concentrations and potential therapeutic utility (Pope et al. J Clin Pharmacol. 2004; 44:1132-1142; Zhang et al. Clin Pharmacol Ther. 1992; 51:647-55; and Kimiskidis et al. Methods Find Exp Clin Pharmacol. 1999; 21:673-8). While difficult to extrapolate human dose requirements from animal data, it appears that dextromethorphan doses higher than typically used for antitussive effects (60 to 120 mg/day, oral), and those used in most previous neuroprotection trials, are required for neuroprotection (Gredal et al. Acta Neurol Scand. 1997; 96:8-13; Albers et al. Stroke. 1991; 22:1075-7; and Dematteis et al. Fundam Clin Pharmacol. 1998; 12:526-37). However, in the trial with Huntington's disease patients, plasma concentrations were undetectable in some patients after dextromethorphan doses that were up to 8 times the maximum antitussive dose (Walker et al. Clin Neuropharmacol. 1989; 12:322-30).

One method for increasing the central bioavailability of dextromethorphan is to coadminister the specific and reversible CYP2D6 inhibitor, quinidine, to protect dextromethorphan from extensive first-pass elimination via the cytochrome P4502D6 enzyme (Zhang et al. Clin Pharmacol Ther. 1992; 51:647-55). This approach serves to enhance the exposure to dextromethorphan and limit the exposure to dextrorphan, which may itself be beneficial. While this active metabolite is partially responsible for the neuroprotective effects in some models (Steinberg et al. Neurosci Lett. 1988b; 89:193-197; Trescher et al. Brain Res Dev Brain Res. 1994; 83:224-32; and Kim et al. Life Sci. 2003a; 72:769-83), its action as a more potent phencyclidine (PCP)-like uncompetitive NMDA receptor antagonist is also associated with psychotomimetic disturbances (Dematteis et al. Fundam Clin Pharmacol. 1998; 12:526-37; Albers et al. Stroke. 1995; 26:254-258; and Szekely et al. Pharmacol Biochem Behav. 1991; 40:381-386). Given the robust preclinical evidence for neuroprotective effects of dextromethorphan, strategies that increase the drug's central bioavailability may hold promise for the treatment of various acute and degenerative neurological disorders.

An impressive preclinical body of evidence has proven that dextromethorphan has significant neuroprotective properties in many in vitro and in vivo models of central nervous system injury (Trube et al. Epilepsia. 1994; 35 Suppl 5:S62-7). Dextromethorphan possesses anti-excitotoxic properties in models of NMDA and glutamate neurotoxicity (Choi et al. J Pharmacol Exp Ther. 1987; 242:713-20). These are believed to be functionally related to its neuroprotective effects in models of focal and global ischemia, hypoxic injury, glucose deprivation, traumatic brain and spinal cord injury, as well as seizure paradigms (Collins et al. Ann Intern Med. 1989; 110:992-1000; Bokesch et al. Anesthesiology. 1994; 81:470-7; and Golding et al. Mol Chem Neuropathol. 1995; 24:137-50).

Recently, dextromethorphan has also been shown to inhibit microglial activation via a novel mechanism that appears unrelated to NMDA receptor antagonism (Liu et al. J Pharmacol Exp Ther. 2003; 305:212-8). This important anti-inflammatory action is proposed to underlie the drug's protection of dopamine neurons in Parkinson's disease models (Zhang et al. Faseb J. 2004; 18:589-91), and could possibly have significant heuristic application in Alzheimer's disease against beta-amyloid-induced microglial activation (Rosenberg. Int Rev Psychiatry. 2005; 17:503-514). Finally, the inhibition of 5-HT uptake by dextromethorphan has been implicated in its protective effect against PCA-induced 5-HT depletion and neurotoxicity (Narita et al. Eur J Pharmacol. 1995; 293:277-80). Dextromethorphan has been established to decrease neuronal damage and improve biochemical as well as neurologic outcome in a variety of preclinical investigations.

Dextromethorphan attenuated morphological and chemical evidence of neuronal damage in glutamate toxicity models (DeCoster et al. receptor-mediated neuroprotection against glutamate toxicity in primary rat neuronal cultures. Brain Res. 1995; 671:45-53; and Choi et al. J Pharmacol Exp Ther. 1987; 242:713-20) as well as the loss of vulnerable hippocampal (CA1) neurons in seizure (Kim et al. Neurotoxicology. 1996; 17:375-385) and global ischemia models (Bokesch et al. Anesthesiology. 1994; 81:470-7). Dextromethorphan decreased cerebral infarct size, areas of severe neocortical ischemic damage, and cortical edema after ischemia and reperfusion (Steinberg et al. Stroke. 1988a; 19:1112-1118; Ying et al. Zhongguo Yao Li Xue Bao. 1995; 16:133-6; and Britton et al. Life Sci. 1997; 60:1729-40). For example, dextromethorphan decreased the incidence of frank cerebral infarction in a brain hypoxia-ischemia model (Prince et al. Neurosci Lett. 1988; 85:291-296). In in vitro hypoxia models, dextromethorphan reduced neuronal loss and dysfunction, manifest in a decreased amplitude of the anoxic depolarization (Goldberg et al. Neurosci Lett. 1987; 80:11-5; Luhmann et al. Neurosci Lett. 1994; 178:171-4). However, neuroprotective effects of dextromethorphan are not limited to hypoxic injury.

Dextromethorphan has also attenuated in vitro morphological and chemical evidence of acute glucose deprivation (Monger et al. Brain Res. 1988; 446:144-8). An effect on regional cerebral blood flow (rCBF) was suggested to contribute to the neuroprotective action of dextromethorphan in transient focal ischemia, since dextromethorphan attenuated the sharp, post-ischemic rise in rCBF during reperfusion in the ischemic core and improved delayed hypoperfusion (Steinberg et al. Neurosci Lett. 1991; 133:225-8). A comparable attenuation of post-ischemic hypoperfusion was found with dextromethorphan in incomplete global cerebral ischemia (Tortella et al. Brain Res. 1989b; 482:179-183). Furthermore, there was strong evidence of a correlated improvement in brain function, as dextromethorphan facilitated recovery of the somatosensory evoked potential (Steinberg et al. Neurosci Lett. 1991; 133:225-8), and attenuated electroencephalographic (EEG) dysfunction in these and other ischemia studies (Ying et al. Zhongguo Yao Li Xue Bao. 1995; 16:133-6; Tortella et al. Brain Res. 1989b; 482:179-183). This is consistent with findings of improved neurological function in focal ischemia (Schmid-Elsaesser et al. Exp Brain Res. 1998; 122:121-7; and Tortella et al. J Pharmacol Exp Ther. 1999; 291:399-408).

Similarly, the reduction in hippocampal damage in global ischemia with dextromethorphan seemed to be the basis of improvement in spatial learning and memory (Block et al. Brain Res. 1996; 741:153-9). In brain and spinal cord injury models, dextromethorphan reduced histological and biochemical damage (Duhaime et al. J Neurotrauma. 1996; 13:79-84; Topsakal et al. Neurosurg Rev. 2002; 25:258-66), blocked traumatic spreading depression limiting the spread of traumatic injury (Church et al. J Neurotrauma. 2005; 22:277-90), and also improved the bioenergetic state (Golding et al. Mol Chem Neuropathol. 1995; 24:137-50). Dextromethorphan prevented the in vivo neurodegeneration of nigral dopamine neurons caused by 1-methyl-4-phenyl-1,2,3,6-tetrahydropyridine (MPTP) (Zhang et al. Faseb J. 2004; 18:589-91), and methamphetamine (METH) (Thomas et al. Brain Res. 2005; 1050:190-8) in models of Parkinson's disease via a proposed reduction in microglial activation and associated intracellular reactive oxygen species (ROS). Analogous in vitro studies showed that dextromethorphan reduced glutamate toxicity of dopamine neurons (Vaglini et al. Brain Res. 2003; 973:298-302), as well as inflammation or microglial mediated degeneration of dopamine neurons induced by lipopolysaccharide (LPS) and MPTP, even at very low concentrations of dextromethorphan (Zhang et al. Faseb J. 2004; 18:589-91; and Li et al. Faseb J. 2005a; 19:489-96). Finally, dextromethorphan protected against the 5-HT depleting effects of PCA in two studies (Narita et al. Eur J Pharmacol. 1995; 293:277-80; and Finnegan et al. Brain Res. 1991; 558:109-111), but failed to do so in a third study (Farfel et al. J Pharmacol Exp Ther. 1995; 272:868-75). Dextromethorphan attenuated the PCA induced reduction of 5-HT and its metabolite 5-hydroxyindoleacetic acid (5-HIAA) particularly in striatum (Finnegan et al. Brain Res. 1991; 558:109-111).

This above-referenced work demonstrates that dextromethorphan possesses important neuroprotective properties, and points to potential therapeutic utility of the agent for the treatment of various neurological disorders. These include stroke, epilepsy, post-anoxic brain injury, traumatic brain and spinal cord injury, Parkinson's disease, and other neurodegenerative diseases (Collins et al. Ann Intern Med. 1989; 110:992-1000; Mattson. Neuromolecular Med. 2003; 3:65-94; and Wersinger et al. Curr Med Chem. 2006; 13:591-602). Dextrorphan, the main active metabolite of dextromethorphan, was found to be neuroprotective in many of the same studies as dextromethorphan, particularly glutamate/NMDA toxicity and ischemia models (Steinberg et al. Neurosci Lett. 1988b; 89:193-197; and Choi et al. J Pharmacol Exp Ther. 1987; 242:713-20). This is to be expected considering that dextrorphan has a similar although not identical pharmacological profile, acting at many of the same sites as dextromethorphan, though with different potencies. For example, dextrorphan is a more potent NMDA receptor antagonist than dextromethorphan (Trube et al. Epilepsia. 1994; 35 Suppl 5:S62-7). Conversely, dextromethorphan is a more potent blocker of voltage-gated calcium channels, and has been found to have a slightly greater affinity for sigma-1 receptors than dextrorphan in some studies (Walker et al. Pharmacol Rev. 1990; 42:355-402; and Taylor et al. In: Kamenka J M, Domino E F, eds. Multiple Sigma and PCP Receptor Ligands: Mechanisms for Neuromodulation and Neuroprotection? Ann Arbor, Mich.: NPP Books; 1992:767-778).

The relative neuroprotective efficacies determined in the different experiments appear to be related to differences in receptor mechanisms. Thus, dextrorphan's greater neuroprotective rank order potency compared to dextromethorphan against acute glutamate toxicity correlated with rank order for competition against [3FI]MK-801 binding to the PCP site, suggesting action via the uncompetitive site within the NMDA-operated cation channel (Berman et al. J Biochem Toxicol. 1996; 11:217-26). On the other hand, dextromethorphan appeared to be a more potent neuroprotectant than dextrorphan in a kainic acid (KA)-induced seizure model (Kim et al. Life Sci. 2003a; 72:769-83). In this paradigm, a selective sigma-1 receptor antagonist blocked dextromethorphan's neuroprotective action to a greater extent than the neuroprotective action of dextrorphan, thus implicating the sigma-1 receptor in the protective mechanism. In vitro and in vivo neuroprotection with dextromethorphan occurred in comparable concentration ranges (Choi et al. J Pharmacol Exp Ther. 1987; 242:713-20; Steinberg et al. Neurol Res. 1993; 15:174-80).

Generally, in vitro protective properties were evident at concentrations as low as 10 to 15 microM, with almost complete protection obtainable at 100 microM (Choi. Brain Res. 1987; 403:333-6; Goldberg et al. Neurosci Lett. 1987; 80:11-5; Monyer et al. Brain Res. 1988; 446:144-8; and Berman et al. J Pharmacol Exp Ther. 1999; 290:439-44). An exception to this was the very low dextromethorphan concentrations needed to inhibit microglial activation and inflammatory damage of dopamine neurons: micro- (1 to 10 microM) and femtomolar concentrations had equal efficacy, while nano- and picomolar quantities showed no protective effects (Liu et al. J Pharmacol Exp Ther. 2003; 305:212-8; Zhang et al. Faseb J. 2004; 18:589-91; and Li et al. Faseb J. 2005a; 19:489-96). In vivo neuroprotective dose ranges were typically 10 to 80 mg/kg administered via various routes: 10 to 80 mg/kg intraperitoneal (IP), 12.5 to 75 mg/kg oral (P0), 10 to 24 mg/kg subcutaneous (SC), and a 10 to 20 mg/kg intravenous (IV) loading dose, followed by a 5 to 15 mg/kg/h infusion. In a single study, lower IV doses of 0.156 to 10 mg/kg were used (Tortella et al. J Pharmacol Exp Ther. 1999; 291:399-408).

Steinberg et al. demonstrated in a rabbit transient focal cerebral ischemia model that dextromethorphan reduced neocortical ischemic neuronal damage and edema when adequate plasma and brain levels were achieved (Steinberg et al. Neurol Res. 1993; 15:174-80). In non-ischemic animals, dextromethorphan concentrated 7 to 30 fold in brain versus plasma, and brain levels were highly correlated with plasma levels. Plasma levels ≥500 ng/ml and brain levels ≥10,000 ng/g, or about 37 microM, were neuroprotective. While a therapeutic time window for neuroprotection has not been determined for dextromethorphan in humans, findings in preclinical ischemia models have provided some insight in this regard. Dextromethorphan was administered pre- and post-treatment in the diverse preclinical analyses. Up to 1 hour delayed treatment was found to be beneficial in models of transient focal ischemia (Steinberg et al. Neurosci Lett. 1988b; 89:193-197; and Steinberg et al. Neurol Res. 1993; 15:174-80). This corresponds to preclinical findings for other NMDA receptor antagonists as neuroprotective drugs, which show an early window of therapeutic activity that does not exceed 1 to 2 hours (Sagratella. Pharmacol Res. 1995; 32:1-13).

Dextromethorphan possesses inhibitory properties on oxygen free-radical mediated membrane lipid peroxidation (Topsakal et al. Neurosurg Rev. 2002; 25:258-66), one of the early or acute mechanisms of neuronal damage linked to NMDA receptor activation and calcium influx (Sagratella. Pharmacol Res. 1995; 32:1-13). However, it has also been demonstrated that dextromethorphan requires more prolonged administration to achieve neuroprotection. For example, continuous perfusion of dextromethorphan up to 4 hours after ischemic insult was necessary for maximum efficacy against focal ischemic damage (Steinberg et al. Neuroscience. 1995; 64:99-107). Analogously, multiple dose treatment paradigms were used by other investigators in models of focal ischemia (Britton et al. Life Sci. 1997; 60:1729-40; and Tortella et al. J Pharmacol Exp Ther. 1999; 291:399-408). This suggests an effect of dextromethorphan on delayed neuronal damage. Dextromethorphan's various non-NMDA receptor-related mechanisms, such as effects on voltage-gated calcium conductances and its capability to decrease glutamate release (Annels et al. Brain Res. 1991; 564:341-343), have been proposed to account for this (Sagratella. Pharmacol Res. 1995; 32:1-13). It has been concluded that dextromethorphan shows a broader spectrum of neuroprotective activities than other NMDA receptor antagonists (Sagratella. Pharmacol Res. 1995; 32:1-13).

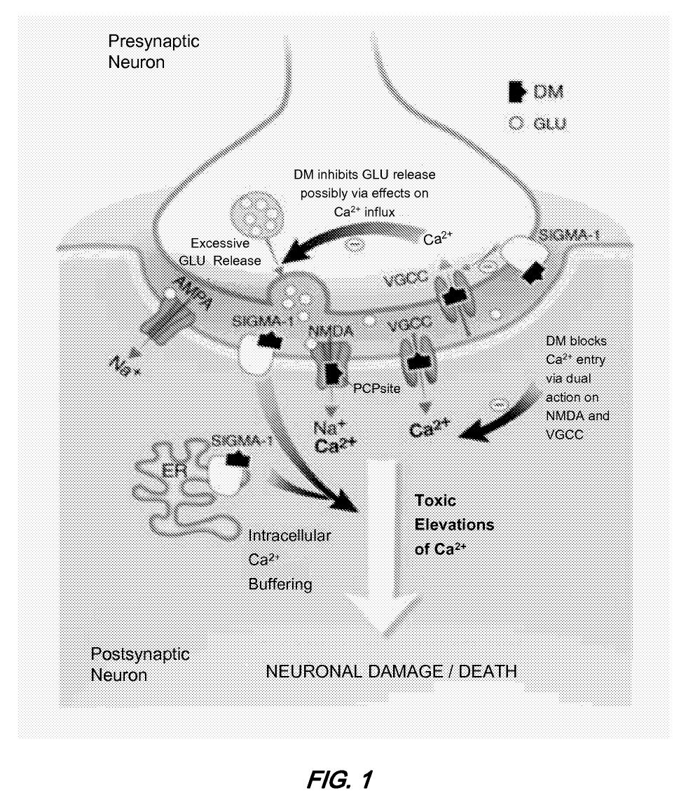

Dextromethorphan has a complex central nervous system pharmacology that is not yet fully elucidated. It has both high and low affinity binding sites related to multiple receptor targets, as well as ion channel and proposed transporter effects, which are thought to contribute to its diverse neuroprotective actions in a variety of neuronal injury models (FIG. 1) (Jaffe et al. Neurosci Lett. 1989; 105:227-32; Zhou et al. Eur J Pharmacol. 1991; 206:261-269; Meoni et al. Br J Pharmacol. 1997; 120:1255-1262; and Trube et al. Epilepsia. 1994; 35 Suppl 5:S62-7). Notably, dextromethorphan's neuroprotective properties in many central nervous system injury models appear functionally related to its anti-excitotoxic effects, as outlined above. Glutamate induced neurotoxicity, and in particular activation of the NMDA subtype of the glutamate receptor, appears to be the common pathway by which a variety of pathogenic processes such as ischemia, hypoxia, hypoglycemia, or prolonged seizures can produce neuronal cell death (Collins et al. Ann Intern Med. 1989; 110:992-1000). Excitotoxic processes have also been implicated in traumatic brain and spinal cord injury, as well as neurodegenerative diseases (Mattson. Neuromolecular Med. 2003; 3:65-94).

Impairment of brain energy metabolism followed by depolarization causes the release of excessive amounts of glutamate into the extracellular space and impairs glutamate reuptake mechanisms, resulting in over-activation of NMDA receptors. This leads to an influx of sodium chloride and water which causes acute neuronal swelling and injury, and calcium which leads to delayed and more permanent damage (Collins et al. Ann Intern Med. 1989; 110:992-1000). Some specific events triggered by toxic elevations of cytosolic free calcium include the activation of intracellular proteases, lipases, and endonucleases, as well as the generation of free radicals (Collins et al. Ann Intern Med. 1989; 110:992-1000). An involvement of NMDA receptors and voltage-gated calcium channels in excitotoxicity-induced elevation of intracellular calcium has been established (Cho. J Neurosci. 1987b; 7:369-379; Choi. Cerebrovasc Brain Metab Rev. 1990; 2:105-147). Thus, the primary mechanisms implicated in the neuroprotective effects of dextromethorphan are low-affinity uncompetitive NMDA receptor antagonism (Tortella et al. Trends Pharmacol Sci. 1989a; 10:501-7; Chou et al. Brain Res. 1999; 821:516-9; and Trube et al. Epilepsia. 1994; 35 Suppl 5:S62-7), blockade of voltage-gated calcium channel conductances (Jaffe et al. Neurosci Lett. 1989; 105:227-32; and Church et al. Neurosci Lett. 1991; 124:232-4), and high-affinity sigma-1 receptor agonist activity (Chou et al. Brain Res. 1999; 821:516-9; Zhou et al. Eur J Pharmacol. 1991; 206:261-269; and Maurice et al. Brain Res Brain Res Rev. 2001; 37:116-32). Additionally, dextromethorphan has been shown to decrease potassium-stimulated glutamate release in brain slices (Annels et al. Brain Res. 1991; 564:341-343). All of these mechanisms, which serve to decrease both the release and harmful effects of glutamate, could interrupt the pathogenic excitotoxic cascade at various points (FIG. 1).

Over a decade ago, NMDA receptor antagonism was suggested to be the predominant mechanism underlying neuroprotective/anticonvulsant properties of dextromethorphan (Trube et al. Epilepsia. 1994; 35 Suppl 5:S62-7). This is supported by findings in glutamate toxicity models, particularly the demonstration that neuroprotective potency correlated with the rank order for competition against [₃H]MK801 binding to the site within the NMDA-operated cation channel (Berman et al. J Biochem Toxicol. 1996; 11:217-26). However, attempts to attribute neuroprotective activity of dextromethorphan purely to NMDA receptor antagonism are complicated by its relatively low-affinity for that site (Tortella et al. Trends Pharmacol Sci. 1989a; 10:501-7; Chou et al. Brain Res. 1999; 821:516-9), as well as by inconsistent findings regarding its ability to prevent glutamate neurotoxicity (Lesage et al. Synapse. 1995; 20:156-64).

Dextromethorphan has been shown to have a broader spectrum of neuroprotective effects compared with other NMDA receptor antagonists (Sagratella. Pharmacol Res. 1995; 32:1-13), as evidenced by the drug's comparatively longer therapeutic time window in focal ischemia (Steinberg et al. Neuroscience. 1995; 64:99-107), and its ability to inhibit delayed neuronal death in global ischemia (Bokesch et al. Anesthesiology. 1994; 81:470-7). It is therefore apparent that mechanisms that may include but are not limited to NMDA receptor antagonism contribute to dextromethorphan's neuroprotective actions, for example the drug's blockade of voltage-gated calcium channels and dextromethorphan's capability to decrease glutamate release, thereby preventing glutamate's action at non-NMDA receptors (Sagratella. Pharmacol Res. 1995; 32:1-13).

Dextromethorphan has been shown to block both NMDA receptor-operated and voltage-gated calcium channels (Jaffe et al. Neurosci Lett. 1989; 105:227-32; and Carpenter et al. Brain Res. 1988; 439:372-5), and to attenuate NMDA- and potassium-evoked increases in cytosolic free calcium concentration in neurons (Church et al. Neurosci Lett. 1991; 124:232-4). These effects occurred at neuroprotective concentrations of dextromethorphan, and it was suggested that the drug's unique ability to inhibit calcium influx via dual routes could result in possible additive or synergistic neuroprotective effects (Jaffe et al. Neurosci Lett. 1989; 105:227-32; and Church et al. Neurosci Lett. 1991; 124:232-4). Furthermore, presynaptic inhibition of voltage-gated calcium channels (VGCC) is suggested to underlie dextromethorphan's reduction of calcium-dependent glutamate release (Annels et al. Brain Res. 1991; 564:341-343). Calcium antagonism and inhibition of glutamate release have been implicated as potential neuroprotective mechanisms in global ischemia and hypoxic injury models (Bokesch et al. Anesthesiology. 1994; 81:470-7; Luhmann et al. Neurosci Lett. 1994; 178:171-4; and Block et al. Neuroscience. 1998; 82:791-803).

It has been demonstrated that dextromethorphan improves cerebral blood flow (CBF) in focal and global ischemia, but not in the normal brain, in such a way that it is thought to contribute to its neuroprotective action (Steinberg et al. Neurosci Lett. 1991; 133:225-8; and Tortella et al. Brain Res. 1989b; 482:179-183).

While the underlying mechanism(s) remain to be elucidated, an attractive suggestion has been that dextromethorphan's effect on CBF may result from blockade of VGCCs located on cerebral blood vessels resulting in vasodilation (Britton et al. Life Sci. 1997; 60:1729-40). Such an action, primarily in ischemic brain regions, could account for dextromethorphan's attenuation of post-ischemic delayed hypoperfusion (Steinberg et al. Neurosci Lett. 1991; 133:225-8; Tortella et al. Brain Res. 1989b; 482:179-183; and Schmid-Elsaesser et al. Exp Brain Res. 1998; 122:121-7). However, this does not explain dextromethorphan's initial reduction of the sharp, post-ischemic rise in regional CBF in the ischemic core during reperfusion, which was observed in a focal ischemia model (Steinberg et al. Neurosci Lett. 1991; 133:225-8). This attenuation of initial hyperemia, however, was not found by all investigators (Schmid-Elsaesser et al. Exp Brain Res. 1998; 122:121-7). In any case, the mechanism is not known, and it is possible that the alterations in CBF seen with dextromethorphan may be secondary to its prevention of excitotoxicity with preserved autoregulation and coupling of blood flow to intact neuronal metabolism (Britton et al. Life Sci. 1997; 60:1729-40; and Steinberg et al. Neurosci Lett. 1991; 133:225-8).

Sigma-1 receptor agonist action is considered to be another important neuroprotective mechanism of dextromethorphan (Chou et al. Brain Res. 1999; 821:516-9). A sigma-1 receptor-related mechanism was implicated in kainic acid-induced seizure models (Kim et al. Life Sci. 2003a; 72:769-83; and Shin et al. Br J Pharmacol. 2005a; 144:908-18), and a traumatic brain injury model (Church et al. J Neurotrauma. 2005; 22:277-90), in which sigma-1 receptor antagonists reversed the protective effects of dextromethorphan. DeCoster et al. found a positive correlation between neuroprotective potency and sigma-1 site affinity in a glutamate toxicity model (DeCoster et al. Brain Res. 1995; 671:45-53). It must be kept in mind that the majority of sigma-1 ligands tested in this correlational study, including dextromethorphan, also have a significant to moderate affinity for the NMDA/PCP site (DeCoster et al. Brain Res. 1995; 671:45-53). However, selective sigma ligands with negligible affinity for the NMDA receptor complex also have notable in vitro neuroprotective efficacy in hypoxia/hypoglycemia models, while being less efficient against glutamate/NMDA toxicity (Maurice et al. Prog Neuropsychopharmacol Biol Psychiatry. 1997; 21:69-102; Maurice. Drug News Perspect. 2002; 15:617-625).

Further, selective sigma receptor agonists reduced neuronal damage in some but not other in vivo models of cerebral ischemia (Maurice et al. Prog Neuropsychopharmacol Biol Psychiatry. 1997; 21:69-102). The precise role and physical nature of sigma-1 receptors in the central nervous system remains unclear. Sigma-1 sites are enriched in the plasma membrane of neuronal cells like classic proteic receptors, but they are also located on intracellular membrane organelles or dispersed throughout the cytoplasm (Maurice et al. Brain Res Brain Res Rev. 2001; 37:116-32). Neurosteroids and neuropeptide Y (NPY) have been proposed to be potential endogenous sigma ligands (Roman et al. Eur J Pharmacol. 1989; 174:301-302; Ault et al. Schizophr Res. 1998; 31:27-36; Nuwayhid et al. J Pharmacol Exp Ther. 2003; 306:934-940; and Maurice et al. Jpn J Pharmacol. 1999; 81:125-55). Later experiments established that sigma and NPY receptor effects more likely converged at the level of signaling (Hong et al. Eur J Pharmacol. 2000; 408:117-125). Neurosteroids thus remain the best candidate endogenous ligands for sigma receptors.

Sigma receptors appear to serve important neuromodulatory roles regulating the release of various neurotransmitters (Maurice et al. Brain Res Brain Res Rev. 2001; 37:116-32; and Werling et al. In: Matsumoto R R, Bowen W D, Su T P, eds. Sigma Receptors: Chemistry, Cell Biology and Clinical Implications. Kluwer Academic Publishers; 2006). Importantly, sigma-1 receptor agonists modulate extracellular calcium influx and intracellular calcium mobilization (Maurice et al. Brain Res Brain Res Rev. 2001; 37:116-32). It is hypothesized that the neuroprotective action of selective sigma ligands may relate to an indirect inhibition of ischemic-induced presynaptic glutamate release (Maurice et al. Prog Neuropsychopharmacol Biol Psychiatry. 1997; 21:69-102). Therefore, the previously mentioned reduction of glutamate release by dextromethorphan (Annels et al. Brain Res. 1991; 564:341-343) could be accounted for by sigma-related inhibition of VGCC dependent synaptic release via a putative G-protein-sigma-receptor coupled mechanism, although this remains speculative (Maurice et al. Prog Neuropsychopharmacol Biol Psychiatry. 1997; 21:69-102; and Maurice et al. Jpn J Pharmacol. 1999; 81:125-55).

On the other hand, selective sigma ligands could be exerting their neuroprotective properties by acting through a putative postsynaptic and/or presynaptic intracellular target protein implicated in intracellular buffering of glutamate-induced calcium flux (Maurice et al. Brain Res Brain Res Rev. 2001; 37:116-32; Maurice et al. Prog Neuropsychopharmacol Biol Psychiatry. 1997; 21:69-102; and DeCoster et al. Brain Res. 1995; 671:45-53). An indirect modulation of NMDA receptor activity is also involved in the neuroprotective effects of certain selective sigma ligands, although the neuroprotective effects of dextromethorphan have been related to a direct antagonism of the NMDA receptor complex (Maurice et al. Prog Neuropsychopharmacol Biol Psychiatry. 1997; 21:69-102; and DeCoster et al. Brain Res. 1995; 671:45-53).

FIG. 1 illustrates the principal mechanisms by which dextromethorphan is proposed to exert its neuroprotective effects at the cellular level. Some neuroprotective action in several preclinical models, as well as side effects, may be attributable to dextromethorphan's active metabolite dextrorphan. Protective effects of both dextrorphan and dextromethorphan have been chiefly noted in glutamate toxicity (Choi et al. J Pharmacol Exp Ther. 1987; 242:713-20; Berman et al. J Biochem Toxicol. 1996; 11:217-26), as well as in vitro and in vivo ischemia models (Steinberg et al. Neurosci Lett. 1988b; 89:193-197; Goldberg et al. Neurosci Lett. 1987; 80:11-5; and Monyer et al. Brain Res. 1988; 446:144-8).

As discussed above, dextrorphan acts on many of the same sites as dextromethorphan but with different affinities or potencies. While specific reported affinities for dextromethorphan and dextrorphan at the site within the NMDA receptor-operated cation channel vary, it is generally agreed that dextrorphan has a distinctly greater affinity than dextromethorphan (Chou et al. Brain Res. 1999; 821:516-9; and Sills et al. Mol Pharmacol. 1989; 36:160-165), and dextrorphan has been shown to be about 8 times more potent than dextromethorphan as an NMDA receptor antagonist (Trube et al. Epilepsia. 1994; 35 Suppl 5:S62-7). Dextrorphan's greater affinity at the NMDA receptor is implicated in greater neuroprotective effects of the agent compared to dextromethorphan in some models (Goldberg et al. Neurosci Lett. 1987; 80:11-5; Monyer et al. Brain Res. 1988; 446:144-8; and Berman et al. J Biochem Toxicol. 1996; 11:217-26) while it is also associated with psychotomimetic disturbances (Dematteis et al. Fundam Clin Pharmacol. 1998; 12:526-37; Albers et al. Stroke. 1995; 26:254-258; and Szekely et al. Pharmacol Biochem Behav. 1991; 40:381-386).

Since NMDA antagonist actions can be extremely complex at the receptor level, further studies are needed to elucidate whether low-affinity uncompetitive antagonist and/or more potent antagonist receptor actions better provide for neuroprotection. In contrast to dextrorphan, dextromethorphan is more effective at inhibiting calcium uptake in vitro due to a 3 times more potent blockade of voltage-gated calcium flux (Jaffe et al. Neurosci Lett. 1989; 105:227-32; Carpenter et al. Brain Res. 1988; 439:372-5; and Trube et al. Epilepsia. 1994; 35 Suppl 5:S62-7) Both drugs bind sigma-1 receptors and have been shown do so with a similar high affinity (Chou et al. Brain Res. 1999; 821:516-9; and Lemaire et al. In: Kamenka J M, Domino E F, eds. Multiple Sigma and PCP Receptor Ligands: Mechanisms for Neuromodulation and Neuroprotection? Ann Arbor, Mich.: NPP Books; 1992:287-293) or with dextromethorphan having a slightly greater (about 2 times) affinity than dextrorphan (Walker et al. Pharmacol Rev. 1990; 42:355-402; and Taylor et al. In: Kamenka J M, Domino E F, eds. Multiple Sigma and PCP Receptor Ligands: Mechanisms for Neuromodulation and Neuroprotection? Ann Arbor, Mich.: NPP Books; 1992:767-778).

Evidence suggests that dextromethorphan binds the serotonin transporter with high-affinity (Meoni et al. Br J Pharmacol. 1997; 120:1255-1262), which might also confer neuroprotection in some paradigms (Narita et al. Eur J Pharmacol. 1995; 293:277-80), while dextrorphan does not. There may also be other sites at which dextromethorphan or dextrorphan act, and it is unclear if the parent compound and metabolite bind the exact same site within the NMDA receptor-channel complex (LePage et al. Neuropharmacology. 2005; 49:1-16). In this regard, autoradiographic studies show a differential pattern of binding for radiolabeled dextrorphan than for dextromethorphan or the other open channel blockers of the NMDA-operated cation channel, and also different from sigma sites (Roth et al. J Pharmacol Exp Ther. 1996; 277:1823-1836). Such mechanistic differences could account for the differential neuroprotective efficacies of dextromethorphan and dextrorphan in various central nervous system injury models (Kim et al. Life Sci. 2003a; 72:769-83; and Berman et al. J Biochem Toxicol. 1996; 11:217-26).

Protective effects of dextromethorphan clearly go beyond effects of dextrorphan. For instance, in a focal ischemia study, Steinberg et al. suggested that dextromethorphan's neuroprotective action was not mediated by dextrorphan, since dextrorphan plasma and brain levels were lower than neuroprotective levels of dextrorphan in the same model (Steinberg et al. Neurol Res. 1993; 15:174-80). Furthermore, focal administration of dextromethorphan into the brain in one transient cerebral ischemia study was neuroprotective (Ying Neurol Res. 1993; 15:174-80. Zhongguo Yao Li Xue Bao. 1995; 16:133-6). Since CYP2D6 is only expressed at low levels in the brain (Steinberg et al. Neurol Res. 1993; 15:174-80; Tyndale. Drug Metab Dispos. 1999; 27:924-30; Britto et al. Drug Metab Dispos. 1992; 20:446-450), this effect and the in vitro neuroprotective properties of dextromethorphan likely do not involve metabolism to an active metabolite, at least not to the extent accomplished by first-pass, hepatic metabolism in vivo. In this regard, dextromethorphan analogs have also demonstrated protective effects against glutamate in cultured cortical neurons unrelated to the biotransformation of dextromethorphan (Tortella et al. Neurosci Lett. 1995; 198:79-82). Another analog of dextromethorphan known not to form dextrorphan (dimemorfan) protected against seizure-induced neuronal loss with fewer PCP-like side effects (Shin et al. Br J Pharmacol. 2005a; 144:908-18).

Dextromethorphan has been recently discovered to interfere with inflammatory responses that are associated with neurodegeneration in chronic diseases such as Parkinson's disease and Alzheimer's disease (Rosenberg. Int Rev Psychiatry. 2005; 17:503-514; and Wersinger et al. Curr Med Chem. 2006; 13:591-602). This novel mechanism is proposed to underlie dextromethorphan's protection of dopamine neurons in both in vitro and in vivo Parkinson's disease models (Liu et al. J Pharmacol Exp Ther. 2003; 305:212-8; Zhang et al. Faseb J. 2004; 18:589-91; and Thomas et al. Brain Res. 2005; 1050:190-8). Neuroprotective effects in these models are concluded to be unlikely due to action on NMDA receptors (Liu et al. J Pharmacol Exp Ther. 2003; 305:212-8).

Dextromethorphan was found to inhibit the activation of microglia, immune cells of the central nervous system, and their production of ROS. The agent reduced LPS- and MPTP-induced production of proinflammatory factors, including tumor necrosis factor-alpha, prostaglandin E2, nitric oxide, and especially superoxide free radicals (Liu et al. J Pharmacol Exp Ther. 2003; 305:212-8; Zhang et al. Faseb J. 2004; 18:589-91; and Li et al. Faseb J. 2005a; 19:489-96). Specifically, dextromethorphan is proposed to act on reduced nicotinamide adenine dinucleotide phosphate (NADPH) oxidase, the primary enzymatic system in microglia for generation of ROS, since neuroprotection was not observed in NADPH oxidase-deficient animals (Liu et al. J Pharmacol Exp Ther. 2003; 305:212-8; and Li et al. Faseb J. 2005a; 19:489-96). Equal protection occurred at low femto and micromolar, but not nano- and picomolar, concentrations, thus yielding a bimodal reversed W-shape dose-response relationship (Li et al. Faseb J. 2005a; 19:489-96). The meaning of such a complex curve is not clear.

A final protective mechanism of dextromethorphan implicated in a serotonergic neurotoxicity model may be its inhibition of 5-HT uptake (Narita et al. Eur J Pharmacol. 1995; 293:277-80). Dextromethorphan was shown to protect against the 5-HT depleting effects of PCA in two (Narita et al. Eur J Pharmacol. 1995; 293:277-80; and Finnegan et al. Brain Res. 1991; 558:109-111) but not a third study (Farfel et al. J Pharmacol Exp Ther. 1995; 272:868-75). The agent attenuated long-term reduction of 5-HT and its metabolite 5-HIAA in rat striatum and cortex. Dextromethorphan alone produced no significant changes in the concentrations of 5-HT or 5-HIAA after 10 days (Finnegan et al. Brain Res. 1991; 558:109-111).

Since potent and selective sigma receptor ligands did not antagonize PCA-induced neurotoxicity, sigma receptors were not thought to play a significant role (Narita et al. Eur J Pharmacol. 1995; 293:277-80). It is proposed that dextromethorphan exerted its beneficial effects by inhibiting 5-HT uptake (Narita et al. Eur J Pharmacol. 1995; 293:277-80). This conclusion is supported by the following findings. First, acute administration of dextromethorphan decreases the 5-HIAA/5-HT ratio in brain, an effect which is well known to occur with 5-HT uptake inhibitors (Henderson et al. Brain Res. 1992; 594:323-326). Second, dextromethorphan is proposed to bind with high affinity, in a sodium-dependent fashion, to the brain serotonin transporter (Meoni et al. Br J Pharmacol. 1997; 120:1255-1262). Finally, action as a weak serotonin reuptake inhibitor (SRI) has been ascribed to dextromethorphan, due to its involvement in serotonin toxicity reactions with monoamine oxidase inhibitors (MAOIs) (Gillman. Br J Anaesth. 2005; 95:434-41; Meoni et al. Br J Pharmacol. 1997; 120:1255-1262).

The potential safety and efficacy of dextromethorphan as a neuroprotective agent have been examined in a limited number of small clinical trials. These have primarily assessed the safety/tolerability of the agent in various patient populations with both acute and chronic neurological disorders. Symptom improvement was demonstrated in some studies. Four studies were designed to evaluate neuroprotection, and two of these found neuroprotective effects (Gredal et al. Acta Neurol Scand. 1997; 96:8-13; and Schmitt et al. Neuropediatrics. 1997; 28:191-7). Studies with negative findings did not utilize doses sufficient for neuroprotection. The largest (N=181) dose-escalation safety and tolerance study of dextromethorphan was conducted in neurosurgery patients undergoing intracranial surgery or endovascular procedures, associated with a high risk of cerebral ischemia (Steinberg et al. J Neurosurg. 1996; 84:860-6). Patients were given oral dextromethorphan (0.8 to 9.64 mg/kg), starting 12 hours prior to surgery and continuing up to 24 hours after surgery. Serum dextromethorphan levels correlated highly with CSF and brain levels. Dextromethorphan concentrated in brain with levels being 68-fold higher than in serum, similar to findings in animals (Steinberg et al. Neurol Res. 1993; 15:174-80; and Wills et al. Pharm Res. 1988; 5:PP1377). The maximum dextromethorphan levels attained were 1514 ng/ml in serum and 92,700 ng/g in brain. In 11 patients, brain and plasma levels of dextromethorphan were comparable to levels that have been shown to be neuroprotective in animal models of cerebral ischemia (serum dextromethorphan ≥500 ng/ml and brain dextromethorphan ≥10,000 ng/g). Frequent adverse events occurring at neuroprotective levels of dextromethorphan included nystagmus, nausea and vomiting, distorted vision, feeling “drunk,” ataxia, and dizziness. All symptoms, even at the highest levels, proved to be tolerable and reversible, and no patient suffered severe adverse reactions.

A few other, smaller studies have examined the role of orally administered dextromethorphan in patients with stroke (N=22 total; dextromethorphan serum levels ranging from 0 to 189 ng/ml) (Albers et al. Stroke. 1991; 22:1075-7; and Albers et al. Clin Neuropharmacol. 1992; 15:509-14) Huntington's disease (N=11; dextromethorphan serum levels ranging from 0 to 280 ng/ml) (Walker et al. Clin Neuropharmacol. 1989; 12:322-30) and amyotrophic lateral sclerosis (N=13; despite high doses, dextromethorphan steady-state plasma levels were detectable in only 1 of 7 patients, with a Cmax of 190 ng/ml) (Hollander et al. Ann Neurol. 1994; 36:920-4). These studies found tolerable adverse events at a variety of doses, ranging from 120 to about 960 mg/day. Common side effects included dizziness, dysarthria, and ataxia at lower doses and hallucinations and fatigue at higher doses. The role of high-dose oral dextromethorphan in patients with amyotrophic lateral sclerosis was evaluated in a phase 1, open-label safety study (N=13) (Hollander et al. Ann Neurol. 1994; 36:920-4). Escalating doses to a maximum tolerable dose of 4.8 to 10 mg/kg/day were given, and patients were maintained on this dose for up to 6 months. The most common adverse events were light-headedness, slurred speech, and fatigue. Side effects were usually tolerable, although they became dose-limiting in most patients. Neuropsychological testing detected no evidence of cognitive dysfunction at high doses in these amyotrophic lateral sclerosis patients (Hollander et al. Ann Neurol. 1994; 36:920-4), which was consistent with findings in a randomized, placebo-controlled safety study of patients with a history of cerebral ischemia (N=12) (Albers et al. Clin Neuropharmacol. 1992; 15:509-14). Overall, the safety trials demonstrate the viability of both long-term and high-dose administration of dextromethorphan to patients with conditions associated with glutamate excitotoxicity (Hollander et al. Ann Neurol. 1994; 36:920-4). Given rapid conversion of dextromethorphan to dextrorphan, it may be that some adverse events encountered with dextromethorphan administration are actually related to dextrorphan.

The safety/tolerability of dextrorphan, the primary metabolite of dextromethorphan, was also assessed in a dose-escalation study with acute ischemic stroke patients (N=67) (Albers et al. Stroke. 1995; 26:254-258). Patients were treated with an intravenous (IV) infusion of dextrorphan within 48 hours of onset of mild-to-moderate hemispheric stroke. There was no difference in neurological outcome at 48 hours between the dextrorphan- and placebo-treated subjects, although the study was not designed to evaluate efficacy. Common transient, reversible, and generally mild to moderate adverse events included nystagmus, nausea, vomiting, somnolence, hallucinations, and agitation. Reversible hypotension was seen with higher loading doses of 200 to 260 mg/h. More severe adverse events such as apnea or deep stupor were observed in patients given the highest doses of dextrorphan. Lower doses (loading doses of 145 to 180 mg, maintenance infusions of 50 to 70 mg/h) were better tolerated and rapidly produced potentially neuroprotective plasma concentrations of dextrorphan (maximum serum levels ranging from 750 to 1000 ng/ml). Dextrorphan has been found to be almost 8 times more potent than dextromethorphan as a NMDA receptor antagonist (Trube et al. Epilepsia. 1994; 35 Suppl 5:S62-7), and to have a much greater affinity for the PCP site in the NMDA receptor complex (Chou et al. Brain Res. 1999; 821:516-9). As could be predicted, the doses tested were associated with well-defined pharmacological effects compatible with blockade of the NMDA receptor (Albers et al. Stroke. 1995; 26:254-258) These findings are consistent with animal studies in which PCP-like effects were observed with dextrorphan but not dextromethorphan (Dematteis et al. Fundam Clin Pharmacol. 1998; 12:526-37; and Szekely et al. Pharmacol Biochem Behav. 1991; 40:381-386), and in which dextromethorphan appeared to have a better therapeutic index at cerebroprotective levels (Steinberg et al. Neurol Res. 1993; 15:174-80).

There is preliminary clinical evidence for a neuroprotective effect of dextromethorphan. Pilot data from a small randomized, placebo-controlled study (N=13) of perioperative brain injury in children undergoing cardiac surgery with cardiopulmonary bypass suggest such an effect (Schmitt et al. Neuropediatrics. 1997; 28:191-7). Dextromethorphan (oral, high-dose 36-38 mg/kg/day, dosing started 24 hours before and ended 96 hours after surgery) reached putative therapeutic levels in plasma (maximal about 550 to 1650 ng/ml) and CSF (285 to 939 ng/ml), and significantly decreased postoperative EEG sharp waves (p=0.02). There were also reduced rates of postoperative periventricular white matter lesions (0/6 dextromethorphan vs. 2/7 placebo) and less pronounced third ventricle postoperative enlargement (diameter 0.112 cm dextromethorphan vs. 0.256 cm placebo; p=0.06), but small sample sizes may have precluded statistical significance. Adverse events were not observed. Reduced EEG sharp wave activity, ventricular enlargement, and the absence of new white matter hyperintense lesions in the dextromethorphan group may be indications of a neuroprotective effect (Schmitt et al. Neuropediatrics. 1997; 28:191-7). However, dissimilarities of treatment groups by chance precluded firm conclusions.

Although amyotrophic lateral sclerosis studies have produced disappointing findings, sub-neuroprotectant doses were employed in these investigations. A randomized, double-blind, placebo-controlled trial with amyotrophic lateral sclerosis patients (N=45) did not demonstrate an improvement in 12-month survival with a relatively low dose of dextromethorphan (150 mg/day; about 2 to 3 mg/kg) (Gredal et al. Acta Neurol Scand. 1997; 96:8-13). Although there was a significantly decreased rate of decline in lower extremity function scores in the dextromethorphan group, baseline differences between the groups precluded firm conclusions. A second 1-year trial (N=49) showed no significant differences in rate of disease progression between dextromethorphan- (1.5 mg/kg/day) and placebo-treated patients (Blin et al. Clin Neuropharmacol. 1996; 19:189-192). Finally, in a third amyotrophic lateral sclerosis study (N=14) no clinical or neurophysiological parameter (relative number of axons, and compound muscle action potentials) improvements were found with dextromethorphan in a 12-week placebo-controlled, crossover study (150 mg/day), followed by an up to 6 months open trial (300 mg/day) (Askmark et al. J Neurol Neurosurg Psychiatry. 1993; 56:197-200). As noted above, preclinical studies have established that considerably higher doses (about 10 to 75 mg/kg, oral) are required for neuroprotective effects.

Symptom improvement with dextromethorphan has been observed in some, but not all studies. A retrospective chart review (N=5) evaluated dextromethorphan (oral 1-2 mg/kg) for severe sub-acute methotrexate (MTX) neurotoxicity (Drachtman et al. Pediatr Hematol Oncol. 2002; 19:319-327). This is a frequent complication of MTX therapy for malignant and inflammatory diseases, the multifactorial pathogenesis of which is thought to involve NMDA receptor activation (Drachtman et al. Pediatr Hematol Oncol. 2002; 19:319-327). Remarkably, dextromethorphan given 1 to 2 weeks after a dose of MTX completely resolved neurological symptoms, including dysarthria and hemiplegia, in all patients. It is possible that dextromethorphan could prevent permanent neurotoxic lesions associated with MTX therapy, but this was not assessed (Drachtman et al. Pediatr Hematol Oncol. 2002; 19:319-327). Two small studies with Parkinson's disease patients (N=22 total) lasting a few weeks showed significant efficacy for symptom improvement at daily doses ranging between 180 and 360 mg (Bonuccelli et al. Lancet. 1992; 340:53; Saenz et al. Neurology. 1993; 43:15). A third study of Parkinson's disease patients (N=21) failed to find symptomatic improvement, but found dose-limiting side effects at 180 mg/day (Montastruc et al. Mov Disord. 1994; 9:242-243). None of these three Parkinson's disease investigations employed neuroprotective methodology. Dextromethorphan also significantly improved levodopa-associated motor complications in two small trials (N=24 total), although with a narrow therapeutic index (Verhagen et al. Neurology. 1998b; 51:203-206; and Verhagen et al. Mov Disord. 1998c; 13:414-417). Interestingly, the researchers co administered dextromethorphan (mean dose 95 to 110 mg/day) with quinidine (100 mg BID) in these trials. In any case, these studies of levodopa-related dyskinesias and motor fluctuations, lasting a few weeks, did not specifically examine neuroprotection. The mentioned open-label trial with Huntington's disease patients (N=11) also found no windows of symptomatic benefit after 4 to 8 weeks of treatment, despite the achievement of a moderately high median peak tolerated dose (410 mg/day) (Walker et al. Clin Neuropharmacol. 1989; 12:322-30). At maximum doses, performance declined on a variety of measures of Huntington's disease (functional rating scales and quantitative exam scores), consistent with dose-related side effects. Oral doses of dextromethorphan did not correlate with serum levels, which varied widely (0 to 280 ng/ml) and were randomly distributed. Nonetheless, the investigators concluded that further trials of dextromethorphan as protective therapy in Huntington's disease may be called for given the proven safety of dextromethorphan in Huntington's disease patients, its salutary effects in animal models of the disease, and the hypothesis that striatal neuronal death in Huntington's disease is mediated by NMDA receptors (Walker et al. Clin Neuropharmacol. 1989; 12:322-30).

Taken together, the favorable safety profile of dextromethorphan, the strong preclinical evidence of neuroprotective effects, the initial positive findings in several clinical studies, and the failure to obtain suitable plasma drug levels in many patients, warrant further trials using strategies that enhance the central bioavailability of dextromethorphan and limit the accumulation of dextrorphan (Pope et al. J Clin Pharmacol. 2004; 44:1132-1142; Zhang et al. Clin Pharmacol Ther. 1992; 51:647-55; and Kimiskidis et al. Methods Find Exp Clin Pharmacol. 1999; 21:673-8).

Preclinical studies have suggested that neuroprotective effects of dextromethorphan are dependent on adequate drug concentrations in the blood reaching the brain. For example, a greater reduction in ischemic neuronal damage was observed with higher plasma levels of dextromethorphan in a rabbit model of transient focal cerebral ischemia (Steinberg et al. Neurol Res. 1993; 15:174-80). In this study, neuroprotective brain levels were greater than 10,000 ng/g. Similarly, other studies have shown a dose-dependent decrease in ischemic or seizure-induced neuronal damage (Kim et al. Neurotoxicology. 1996; 17:375-385; Gotti et al. Brain Res. 1990; 522:290-307; and Yin et al. Zhongguo Yao Li Xue Bao. 1998; 19:223-6), although a clear relationship between dextromethorphan dose and degree of brain protection was not always found (Prince et al. Neurosci Lett. 1988; 85:291-296; and Tortella et al. J Pharmacol Exp Ther. 1999; 291:399-408). Preclinical studies in which neuroprotection was observed utilized oral dextromethorphan doses of about 10 to 75 mg/kg, whereas clinical neuroprotection studies have usually employed lower doses. As in humans, a substantial effect of first-pass metabolism on dextromethorphan bioavailability has been shown in animals, and route-specific effects on the disposition of dextromethorphan and dextrorphan in the plasma and brain must be considered (Wu et al. J Pharmacol Exp Ther. 1995; 274:1431-7).

Several investigators have proposed that the limited benefit seen with dextromethorphan as a neuroprotectant in clinical trials is associated with its rapid metabolism which does not allow the attainment of sufficient systemic drug concentrations (Pope et al. J Clin Pharmacol. 2004; 44:1132-1142; Zhang et al. Clin Pharmacol Ther. 1992; 51:647-55; and Kimiskidis et al. Methods Find Exp Clin Pharmacol. 1999; 21:673-8). As discussed above, in most humans, dextromethorphan undergoes extensive hepatic 0-demethylation to its primary metabolite dextrorphan, which is catalyzed by the polymorphic cytochrome P450 2D6 (CYP2D6). Metabolism is so great that after a single oral dose of dextromethorphan (30 mg), dextromethorphan was not detectable or at the limits of detection in the plasma of extensive metabolizers (N=5), constituting the majority of the population (Schadel et al. J Clin Psychopharmacol. 1995; 15:263-9). Poor metabolizers of dextromethorphan comprise ≤7 percent of the population (Droll et al. Pharmacogenetics. 1998; 8:325-333). Dextrorphan is rapidly glucuronidated and cleared, while dextromethorphan is not conjugated and concentrates in the brain (Pope et al. J Clin Pharmacol. 2004; 44:1132-1142). Steinberg et al. measured brain levels 68-fold higher than serum levels in neurosurgery patients given oral dextromethorphan, and brain levels correlated highly with serum levels (Steinberg et al. J Neurosurg. 1996; 84:860-6). A precise relationship between dextromethorphan dose and plasma or serum concentration has not yet emerged (Walker et al. Clin Neuropharmacol. 1989; 12:322-30; Zhang et al. Clin Pharmacol Ther. 1992; 51:647-55), although Steinberg et al. did observe that higher doses generally increased dextromethorphan serum levels (Steinberg et al. J Neurosurg. 1996; 84:860-6) These complex pharmacokinetics are suggested to explain why even large doses of dextromethorphan (up to 960 mg/day; median 410 mg/day) produced a random distribution of, and in some cases undetectable, dextromethorphan serum concentrations (0 to 280 ng/ml) in Huntington's disease patients (Walker et al. Clin Neuropharmacol. 1989; 12:322-30). Similarly, plasma dextromethorphan was detectable in only 1 of 7 amyotrophic lateral sclerosis patients at steady state (190 ng/ml at 3 months) despite administration of 4.8 to 10 mg/kg/day (median 7 mg/kg/day) of dextromethorphan in a safety study (Hollander et al. Ann Neurol. 1994; 36:920-4). As described, exceptionally high dextromethorphan levels were attained by Steinberg et al. (Steinberg et al. J Neurosurg. 1996; 84:860-6) in neurosurgery patients (maximum 1514 ng/ml in serum and maximum 9.64 mg/kg oral dose), and by Schmitt et al. (Schmitt et al. Neuropediatrics. 1997; 28:191-7) in cardiac surgery patients (maximum 1650 ng/ml in plasma and maximum 38 mg/kg/day oral dose). However, these levels were reached with high, multiple doses administered over days: neurosurgery patients were dosed beginning 12 hours before surgery and up to 24 hours after (Steinberg et al. J Neurosurg. 1996; 84:860-6), while cardiac surgery patients were dosed starting 24 hours before until 96 hours after surgery (Schmitt et al. Neuropediatrics. 1997; 28:191-7). Such dosing regimens are not practical over the long-term, and may not be as well tolerated by patients that are awake and not under intensive care unit conditions (Schmitt et al. Neuropediatrics. 1997; 28:191-7; and Steinberg et al. J Neurosurg. 1996; 84:860-6). Limited systemic delivery of dextromethorphan could thus, at least in part, account for disappointing trial results.

Along these lines, it should further be noted that with the exception of the Schmitt et al. study of patients with perioperative brain injury (Schmitt et al. Neuropediatrics. 1997; 28:191-7) the other clinical trials of sufficient duration to evaluate neuroprotection (all in amyotrophic lateral sclerosis patients) used inadequate mg/kg/day doses based on the existing body of preclinical evidence. In animal in vivo studies, dextromethorphan doses of 10 to 80 mg/kg (administered PO, IP, SC, or IV) were generally associated with neuroprotective efficacy, with the exception of a single study that used lower IV doses (Tortella et al. J Pharmacol Exp Ther. 1999; 291:399-40). In a rabbit focal ischemia model, a 20 mg/kg (IV) loading dose alone was not neuroprotective, unless given with a 10 mg/kg/h maintenance infusion (Steinberg et al. Neuroscience. 1995; 64:99-107). The single clinical study wherein neuroprotective effects were observed used dextromethorphan oral doses between 36 to 38 mg/kg/day (concentrations of about 550-1650 ng/ml maximum in plasma and 285-939 ng/ml in CSF) (Schmitt et al. Neuropediatrics. 1997; 28:191-7). In the other three clinical neuroprotection trials, oral doses of only 1.5 to 6 mg/kg/day were employed, which are about 10 to 20 fold below known neuroprotective doses (Gredal et al. Acta Neurol Scand. 1997; 96:8-13; Blin et al. Clin Neuropharmacol. 1996; 19:189-192; and Askmark et al. J Neurol Neurosurg Psychiatry. 1993; 56:197-200).

Enhancing the central bioavailability of dextromethorphan may increase its therapeutic potential as a neuroprotectant (Pope et al. J Clin Pharmacol. 2004; 44:1132-1142). Dextromethorphan doses needed for neuroprotection are greater than antitussive doses (Albers et al. Stroke. 1991; 22:1075-7; and Dematteis et al. Fundam Clin Pharmacol. 1998; 12:526-37), but due to the pronounced metabolism of dextromethorphan, therapeutic concentrations are not easily achieved by simple dosage adjustment (Zhang et al. Clin Pharmacol Ther. 1992; 51:647-55). Various methods of enhancing dextromethorphan bioavailability have been proposed. For example, since the brain concentration of dextromethorphan is believed to be route dependent, parenteral administration (e.g., intravenous) has been used to avoid the first-pass effect. Similarly, the nasal route has been shown to be a viable alternative in animals, with drug absorption following intravenous profiles (Char et al. J Pharm Sci. 1992; 81:750-2). Nevertheless, oral administration remains the most convenient, particularly for potential treatment of chronic neurological disorders. The most promising strategy for increasing systemically available dextromethorphan therefore appears to be the coadministration of the specific and reversible CYP2D6 inhibitor quinidine (Pope et al. J Clin Pharmacol. 2004; 44:1132-1142; Zhang et al. Clin Pharmacol Ther. 1992; 51:647-55; and Schadel et al. J Clin Psychopharmacol. 1995; 15:263-9). As discussed above, quinidine administration protects dextromethorphan from metabolism after oral dosing, and can convert subjects with the extensive metabolizer to the poor metabolizer phenotype. This results in elevated and prolonged dextromethorphan plasma profiles, increasing the drug's likelihood of reaching neuronal targets (Pope et al. J Clin Pharmacol. 2004; 44:1132-1142). This approach also improves the predictability in dextromethorphan plasma levels, as a strong linear relationship was observed between dextromethorphan dose and plasma concentration, when quinidine was coadministered with increasing doses of dextromethorphan (Zhang et al. Clin Pharmacol Ther. 1992; 51:647-55). Finally, inhibition of dextromethorphan metabolism limits exposure to dextrorphan (Pope et al. J Clin Pharmacol. 2004; 44:1132-1142), implicated in psychotomimetic reactions and abuse liability (Schadel et al. J Clin Psychopharmacol. 1995; 15:263-9)

The use of quinidine to inhibit the rapid first-pass metabolism of dextromethorphan allows the attainment of potential neuroprotective drug levels in the brain. Pope et al. demonstrated that about 30 mg quinidine is the lowest dose needed to maximally suppress 0-demethylation of dextromethorphan (Pope et al. J Clin Pharmacol. 2004; 44:1132-1142). This dose, 30 mg twice daily (BID) given with 60 mg BID dextromethorphan, increased plasma levels of dextromethorphan 25-fold. In this manner, coadministration of 30 mg of quinidine BID with dextromethorphan in the three unsuccessful amyotrophic lateral sclerosis neuroprotection trials could have readily transformed the inadequate dextromethorphan doses into standard neuroprotective plasma concentrations. Pope et al. further showed that 120 mg daily dextromethorphan (60 mg BID) with quinidine (30 mg BID) resulted in steady state peak plasma levels of 192±45 ng/ml and an AUC0-12 of 1963±609 ng·h/ml (Pope et al. J Clin Pharmacol. 2004; 44:1132-1142).

Given the 68-fold concentration of dextromethorphan in brain found in neurosurgery patients (Steinberg et al. J Neurosurg. 1996; 84:860-6), an estimated brain concentration of 13,100 ng/g (about 48 microM) is achievable. This corresponds to neuroprotective levels established in preclinical in vitro (Choi et al. J Pharmacol Exp Ther. 1987; 242:713-20) and in vivo (Steinberg et al. Neurol Res. 1993; 15:174-80) studies.

A reasonable concern is that the achievement of higher dextromethorphan plasma concentrations, as well as the use of quinidine, may be associated with an increased occurrence of adverse events, particularly in patients with neurological disorders. Clinical studies to date have shown the combination of dextromethorphan and quinidine to be generally well tolerated, although the incidence of adverse events did appear to relate to dextromethorphan dose (Pope et al. J Clin Pharmacol. 2004; 44:1132-1142). Safety evaluations in healthy subjects (Total N=120) showed that daily doses of up to 120 mg dextromethorphan plus 120 mg quinidine administered for 1 week, resulted in mostly mild to moderate adverse events (Pope et al. J Clin Pharmacol. 2004; 44:1132-1142). No difference was found between the extensive and poor metabolizer phenotypes.

The most commonly reported adverse events were headache, loose stool, light-headedness, dizziness, and nausea. No electrocardiographic abnormalities were observed. In particular, there was no clinically significant change in the QTc interval. This is important, because quinidine use has been associated with QTc prolongation and the occurrence of a torsade de pointes based arrhythmia (Grace et al. Quinidine. N Engl J Med. 1998; 338:35-45; and Gowda et al. Int J Cardiol. 2004; 96:1-6). However, the low doses of quinidine required to maximally inhibit dextromethorphan metabolism, and to reach potentially neuroprotective levels of dextromethorphan, are about 10- to 30-fold below the 600- to 1600-mg daily doses routinely used to treat cardiac arrhythmias (Grace et al. N Engl J Med. 1998; 338:35-45). The mentioned studies by Pope et al. (Pope et al. J Clin Pharmacol. 2004; 44:1132-1142) provided the rationale for the proprietary fixed combination product AVP-923 (30 mg dextromethorphan and 30 mg quinidine; Zenvia™) in development by Avanir Pharmaceuticals (San Diego, Calif.). Two phase 3 clinical trials testing AVP-923 for involuntary emotional expression disorder have also shown the dextromethorphan and quinidine combination to be generally well tolerated. In these trials with amyotrophic lateral sclerosis (N=140) (Brooks et al. Neurology. 2004; 63:1364-70) and multiple sclerosis (N=150) (Panitch et al. Ann Neurol. 2006; 59:780-787) patients, daily doses of 60 mg dextromethorphan plus 60 mg quinidine BID given for 1 and 3 months resulted in mean steady state plasma levels of about 100 and 115 ng/ml, respectively. As in healthy subjects, use of AVP-923 in these patients with neurodegenerative disorders, even over a prolonged period, resulted in mostly mild to moderate adverse events. The adverse events reported more frequently with AVP-923 than its components (dextromethorphan and quinidine alone) or placebo were dizziness, nausea, and somnolence. No clinically significant changes were noted in QTc interval.

Overall, the use of low-dose quinidine to increase dextromethorphan bioavailability holds promise as a potential neuroprotective strategy. This approach allows the predictable attainment of neuroprotective levels of dextromethorphan found in preclinical studies, and the dextromethorphan/quinidine combination (e.g., the fixed combination product AVP-923) has been shown to be well tolerated in clinical trials. It was suggested over a decade ago that inhibiting the metabolism of dextromethorphan to its primary active metabolite dextrorphan is unnecessary (Hollander et al. Ann Neurol. 1994; 36:920-4), since dextrorphan was thought to be the more potent uncompetitive NMDA receptor antagonist and protective agent (Choi et al. J Pharmacol Exp Ther. 1987; 242:713-20). However, there is a continuously growing body of evidence which now demonstrates that dextromethorphan itself is neuroprotective via diverse mechanisms beyond uncompetitive NMDA receptor antagonism. In some models of central nervous system injury, dextromethorphan has a greater neuroprotective potency than dextrorphan (Kim et al. Life Sci. 2003a; 72:769-83). This methodology is therefore worthy of exploration in the neuroprotective arena.

A large body of preclinical (Trube et al. Epilepsia. 1994; 35 Suppl 5:S62-7) and clinical evidence (Schmitt et al. Neuropediatrics. 1997; 28:191-7; and Drachtman et al. Pediatr Hematol Oncol. 2002; 19:319-327) demonstrates that dextromethorphan possesses important neuroprotective properties, many of which seem functionally related to its inhibition of excitotoxicity (Bokesch et al. Anesthesiology. 1994; 81:470-7). Diverse mechanisms are implicated, the primary ones being low-affinity, uncompetitive NMDA receptor antagonist (Tortella et al. Trends Pharmacol Sci. 1989a; 10:501-7; Chou et al. Brain Res. 1999; 821:516-9; and Trube et al. Epilepsia. 1994; 35 Suppl 5:S62-7), high-affinity sigma-1 receptor agonist (DeCoster et al. Brain Res. 1995; 671:45-53), and voltage-gated calcium channel antagonist effects (Jaffe et al. Neurosci Lett. 1989; 105:227-32). Dextromethorphan's inhibition of glutamate release is thought to be linked with sigma receptor action (Annels et al. Brain Res. 1991; 564:341-343; and Maurice et al. Prog Neuropsychopharmacol Biol Psychiatry. 1997; 21:69-102). Notably, the agent uniquely inhibits calcium influx via multiple routes, with possible additive or synergistic neuroprotective effects (Jaffe et al. Neurosci Lett. 1989; 105:227-32; and Church et al. Neurosci Lett. 1991; 124:232-4).

Dextromethorphan is generally well tolerated in humans, and the use of high doses over prolonged periods has been shown to be feasible in patients with conditions associated with excitotoxic injury (Walker et al. Clin Neuropharmacol. 1989; 12:322-30; Hollander et al. Ann Neurol. 1994; 36:920-4). The use of quinidine to inhibit the metabolism of dextromethorphan allows the attainment of predictable and potentially neuroprotective systemic levels of dextromethorphan (Pope et al. J Clin Pharmacol. 2004; 44:1132-1142). This drug combination was well tolerated in large clinical trials (Pope et al. J Clin Pharmacol. 2004; 44:1132-1142; Brooks et al. Neurology. 2004; 63:1364-70; and Panitch et al. Ann Neurol. 2006; 59:780-787). Together these findings point to the prospective therapeutic utility of dextromethorphan or the dextromethorphan/quinidine combination (e.g., AVP-923) (Brooks et al. Neurology. 2004; 63:1364-70; and Panitch et al. Ann Neurol. 2006; 59:780-787) for the treatment of various acute and chronic neurological disorders.

By pharmacologically inhibiting the release and harmful actions of glutamate via NMDA receptors, as well as blocking multiple routes of calcium influx, dextromethorphan could serve to protect neurons in various neurological disorders in which excitotoxic mechanisms (Collins et al. Ann Intern Med. 1989; 110:992-1000) play a significant pathogenic role. Substantial evidence supports roles for excitotoxicity in acute disorders such as stroke, epileptic seizures, and traumatic brain and spinal cord injury (Mattson. Neuromolecular Med. 2003; 3:65-94).

Given the strong evidence for neuroprotective efficacy of dextromethorphan in preclinical in vivo models of focal and global ischemia (Bokesch et al. Anesthesiology. 1994; 81:470-7; and Steinberg et al. Stroke. 1988a; 19:1112-1118), as well as in vitro models of hypoxic and hypoglycemic injury (Goldberg et al. Neurosci Lett. 1987; 80:11-5; and Monyer et al. Brain Res. 1988; 446:144-8), possible clinical settings in which dextromethorphan may prove to be beneficial include ischemic stroke, cardiac arrest, and neuro- or cardiac-surgical procedures associated with a high risk of cerebral ischemia. The small clinical trial showing possible neuroprotection in perioperative brain injury in children undergoing cardiac surgery with cardiopulmonary bypass provides hope in this regard (Schmitt et al. Neuropediatrics. 1997; 28:191-7) Furthermore, neuroprotective effects found in preclinical models of brain and spinal cord injury (Duhaime et al. J Neurotrauma. 1996; 13:79-84; and Topsakal et al. Neurosurg Rev. 2002; 25:258-66), point to a possible benefit for injury caused by trauma to the central nervous system. A potential factor limiting clinical application would be the need for immediate or prophylactic therapy, as many experimental studies used pretreatment paradigms. However, researchers have reported promising findings of protective efficacy for dextromethorphan administered up to 1 hour after ischemic insult (Steinberg et al. Neurosci Lett. 1988b; 89:193-197; and Steinberg et al. Neurol Res. 1993; 15:174-80). Additionally, in a study of focal cerebral ischemia, 4 hours of dextromethorphan maintenance dosing was required to achieve neuroprotection (Steinberg et al. Neuroscience. 1995; 64:99-107). It has therefore been concluded that dextromethorphan shows a broader spectrum of neuroprotective activities than other NMDA receptor antagonists, which have a narrow therapeutic window (Sagratella. Pharmacol Res. 1995; 32:1-13).

Considerable evidence also supports roles for excitotoxicity in neurodegenerative diseases such as Huntington's disease, amyotrophic lateral sclerosis, Parkinson's disease, and Alzheimer's disease (Mattson. Neuromolecular Med. 2003; 3:65-94; Berman et al. Curr Neurol Neurosci Rep. 2006; 6:281-286; and Van Damme et al. Neurodegener Dis. 2005; 2:147-159). There is a paucity of data that does not allow current inferences about the effects of dextromethorphan/quinidine in these diseases. Only three small amyotrophic lateral sclerosis studies of dextromethorphan evaluated neuroprotective indices, with disappointing results (Gredal et al. Acta Neurol Scand. 1997; 96:8-13; Blin et al. Clin Neuropharmacol. 1996; 19:189-192; and Askmark et al. J Neurol Neurosurg Psychiatry. 1993; 56:197-200). However, these studies used sub-neuroprotective doses of dextromethorphan, and did not ascertain if predictable neuroprotective systemic levels of dextromethorphan were reached. Indeed, high-dose dextromethorphan in an amyotrophic lateral sclerosis safety study did not even result in detectable steady-state plasma and CSF levels in most patients (Hollander et al. Ann Neurol. 1994; 36:920-4). The attainment of potentially neuroprotective levels is now possible with the use of quinidine, and further studies are warranted.

Inflammatory mechanisms, such as activation of microglia, are thought to play a prominent role in the pathogenesis of Parkinson's disease (Wersinger et al. Curr Med Chem. 2006; 13:591-602), Alzheimer's disease (Rosenberg. Int Rev Psychiatry. 2005; 17:503-514), and amyotrophic lateral sclerosis (Guillemin et al. Neurodegener Dis. 2005; 2:166-176). Recent findings with dextromethorphan in Parkinsonian models show that it protects dopamine neurons from inflammation-mediated degeneration in vivo and in vitro (Liu et al. J Pharmacol Exp Ther. 2003; 305:212-8; Zhang et al. Faseb J. 2004; 18:589-91; and Thomas et al. Brain Res. 2005; 1050:190-8). The investigators proposed that dextromethorphan's beneficial effects seen at low concentrations are accounted for by a novel mechanism, specifically inhibition of microglial production of reactive oxygen species (ROS) (Zhang et al. Faseb J. 2004; 18:589-91; and Li et al. Faseb J. 2005a; 19:489-96). More clinical studies of dextromethorphan in Parkinson's disease would be valuable. This is true particularly since there is evidence that dextromethorphan alleviates levodopa-associated motor complications (Verhagen et al. Neurology. 1998b; 51:203-206; and Verhagen et al. Mov Disord. 1998c; 13:414-417) and has helped improve Parkinsonian symptoms in some small studies (Bonuccelli et al. Lancet. 1992; 340:53; Saenz et al. Neurology. 1993; 43:15). Potential neuroprotective properties of dextromethorphan in other conditions involving neurodegenerative inflammatory processes, such as Alzheimer's disease, also appear worthy of pursuit. Provided the unique, pleiotropic mechanism of dextromethorphan, its possible therapeutic applications have only begun to be explored.

### Dextromethorphan for Involuntary Emotional Expression Disorder

The discovery that dextromethorphan can reduce the internal feelings and external symptoms of emotional lability or pseudobulbar affect in some patients suffering from neurodegenerative diseases suggests that dextromethorphan is also likely to be useful for helping some patients suffering from emotional lability due to other causes, such as stroke. other ischemic (low blood flow) or hypoxic (low oxygen supply) events which led to neuronal death or damage in limited regions of the brain, or head injury or trauma as might occur during an automobile, motorcycle, or bicycling accident or due to a gunshot wound.

In addition, the results obtained to date also suggest that dextromethorphan is likely to be useful for treating some cases of emotional lability which are due to administration of other drugs. For example, various steroids, such as prednisone, are widely used to treat autoimmune diseases such as lupus. However, prednisone has adverse events on the emotional state of many patients, ranging from mild but noticeably increased levels of moodiness and depression, up to severely aggravated levels of emotional lability that can impair the business, family, or personal affairs of the patient.

In addition, dextromethorphan in combination with quinidine can reduce the external displays or the internal feelings that are caused by or which accompany various other problems such as “premenstrual syndrome” (PMS), Tourette's syndrome, and the outburst displays that occur in people suffering from certain types of mental illness. Although such problems may not be clinically regarded as emotional lability or involuntary emotional expression disorder, they involve manifestations that appear to be sufficiently similar to emotional lability to suggest that dextromethorphan can offer an effective treatment for at least some patients suffering from such problems.

Dextromethorphan in combination with quinidine can also be used to treat patients suffering from depression, anxiety, or other mood disorders, such as social anxiety disorder, posttraumatic stress disorder), panic disorder, eating disorders (anorexia, bulimia), obsessive-compulsive disorder, and premenstrual dysphoric disorder.

### Pharmaceutical Compositions

One of the significant characteristics of the treatments of preferred embodiments is that the treatments function to reduce symptoms of neurodegenerative disorders, involuntary emotional expression disorder, depression, or anxiety without tranquilizing or otherwise significantly interfering with consciousness or alertness in the patient. As used herein, “significant interference” refers to adverse events that would be significant either on a clinical level (they would provoke a specific concern in a doctor or psychologist) or on a personal or social level (such as by causing drowsiness sufficiently severe that it would impair someone's ability to drive an automobile). In contrast, the types of very minor side effects that can be caused by an over-the-counter drug such as a dextromethorphan-containing cough syrup when used at recommended dosages are not regarded as significant interference.

The magnitude of a prophylactic or therapeutic dose of dextromethorphan in combination with quinidine in the acute or chronic management of symptoms associated with neurodegenerative disorders, involuntary emotional expression disorder, depression, or anxiety can vary with the particular cause of the condition, the severity of the condition, and the route of administration. The dose and/or the dose frequency can also vary according to the age, body weight, and response of the individual patient.

In general, it is preferred to administer the dextromethorphan and quinidine in a combined dose, or in separate doses administered substantially simultaneously. The preferred weight ratio of dextromethorphan to quinidine is about 1:1.5 or less, preferably about 1:1.45, 1:1.4, 1:1.35, or 1:1.3 or less, more preferably about 1:1.25, 1:1.2, 1:1.15, 1:1.1, 1:1.05, 1:1, 1:0.95, 1:0.9, 1:0.85, 1:0.8, 1:0.75, 1:0.7, 1:0.65, 1:0.6, 1:0.55 or 1:0.5 or less. In certain embodiments, however, dosages wherein the weight ratio of dextromethorphan to quinidine is greater than about 1:1.5 may be preferred, for example, dosages of about 1:1.6, 1:1.7, 1:1.8, 1:1.9, 1:2 or greater. Likewise, in certain embodiments, dosages wherein the ratio of dextromethorphan to quinidine is less than about 1:0.5 may be preferred, for example, about 1:0.45, 1:0.4, 1:0.35, 1:0.3, 1:0.25, 1:0.2, 1:0.15, or 1:0.1 or less. Similarly, in certain embodiments, dosages wherein the ratio of dextromethorphan to quinidine is more than about 1:1.5 may be preferred, for example, about 1:1.6, 1:1.7, 1:1.8, 1:1.9, 1:2.0, 1:2.5, 1:3.0, 1:3.5, or 1:4.0 or more. When dextromethorphan and quinidine are administered at the preferred ratio of 1:1.25 or less, it is generally preferred that less than 50 mg quinidine is administered at any one time, more preferably about 45, 40, or 35 mg or less, and most preferably about 30, 25, or 20 mg or less. It may also be preferred to administer the combined dose (or separate doses simultaneously administered) at the preferred ratio of 1:1.25 or less twice daily, three times daily, four times daily, or more frequently so as to provide the patient with a preferred dosage level per day, for example: 60 mg quinidine and 60 mg dextromethorphan per day provided in two doses, each dose containing 30 mg quinidine and 30 mg dextromethorphan; 50 mg quinidine and 50 mg dextromethorphan per day provided in two doses, each dose containing 25 mg quinidine and 25 mg dextromethorphan; 40 mg quinidine and 40 mg dextromethorphan per day provided in two doses, each dose containing 20 mg quinidine and 20 mg dextromethorphan; 30 mg quinidine and 30 mg dextromethorphan per day provided in two doses, each dose containing 15 mg quinidine and 15 mg dextromethorphan; or 20 mg quinidine and 20 mg dextromethorphan per day provided in two doses, each dose containing 10 mg quinidine and 10 mg dextromethorphan. The total amount of dextromethorphan and quinidine in a combined dose may be adjusted, depending upon the number of doses to be administered per day, so as to provide a suitable daily total dosage to the patient, while maintaining the preferred ratio of 1:1.25 or less. These ratios are particularly preferred for the treatment of symptoms associated with neurodegenerative disorders (e.g., Alzheimer's disease, dementia, vascular dementia, amyotrophic lateral sclerosis, multiple sclerosis, and Parkinson's disease), involuntary emotional expression disorder, brain damage (e.g., due to stroke or other trauma), depression, or anxiety, or any of the other indications referred to herein.

In general, the total daily dose for dextromethorphan in combination with quinidine, for the conditions described herein, is about 10 mg or less up to about 200 mg or more dextromethorphan in combination with about 1 mg or less up to about 150 mg or more quinidine; preferably from about 15 or 20 mg to about 65, 70, 75, 80, 85, 90, 95, 100, 110, 120, 130, 140, 150, 160, 170, 180, or 190 mg dextromethorphan in combination with from about 2.5, 5, 7.5, 10, 15, or 20 mg to about 55, 60, 65, 70, 75, 80, 85, 90, 95, 100, 110, 120, 130, or 140 mg quinidine; more preferably from about 25, 30, 35, or 40 mg to about 55 or 60 mg dextromethorphan in combination with from about 25, 30, or 35 mg to about 40, 45, or 50 mg quinidine. In particularly preferred embodiments, the daily dose of dextromethorphan to quinidine is: 20 mg dextromethorphan to 20 mg quinidine; 20 mg dextromethorphan to 30 mg quinidine; 20 mg dextromethorphan to 40 mg quinidine; 20 mg dextromethorphan to 50 mg quinidine; 20 mg dextromethorphan to 60 mg quinidine; 30 mg dextromethorphan to 20 mg quinidine; 30 mg dextromethorphan to 30 mg quinidine; 30 mg dextromethorphan to 40 mg quinidine; 30 mg dextromethorphan to 50 mg quinidine; 30 mg dextromethorphan to 60 mg quinidine; 40 mg dextromethorphan to 20 mg quinidine; 40 mg dextromethorphan to 30 mg quinidine; 40 mg dextromethorphan to 40 mg quinidine; 40 mg dextromethorphan to 50 mg quinidine; 40 mg dextromethorphan to 60 mg quinidine; 50 mg dextromethorphan to 20 mg quinidine; 50 mg dextromethorphan to 30 mg quinidine; 50 mg dextromethorphan to 40 mg quinidine; 50 mg dextromethorphan to 50 mg quinidine; 50 mg dextromethorphan to 50 mg quinidine; 60 mg dextromethorphan to 20 mg quinidine; 60 mg dextromethorphan to 30 mg quinidine; 60 mg dextromethorphan to 40 mg quinidine; 60 mg dextromethorphan to 50 mg quinidine; or 60 mg dextromethorphan to 60 mg quinidine. A single dose per day or divided doses (two, three, four, or more doses per day) can be administered.

Preferably, a daily dose for symptoms associated with neurodegenerative disorders, involuntary emotional expression disorder, depression, or anxiety, or the other conditions referred to herein, is about 20 mg to about 60 mg dextromethorphan in combination with about 20 mg to about 60 mg quinidine, in single or divided doses. Particularly preferred daily dose for symptoms associated with neurodegenerative disorders, involuntary emotional expression disorder, depression, or anxiety, or the other conditions referred to herein, is about 20, 21, 22, 23, 24, 25, 26, 27, 28, 29, or 30 mg dextromethorphan in combination with about 20, 21, 22, 23, 24, 25, 26, 27, 28, 29, or 30 mg quinidine; about 30, 31, 32, 33, 34, 35, 36, 37, 38, 39, or 40 mg dextromethorphan in combination with about 20, 21, 22, 23, 24, 25, 26, 27, 28, 29, or 30 mg quinidine; about 40, 41, 42, 43, 44, 45, 46, 47, 48, 49, or 50 mg dextromethorphan in combination with about 20, 21, 22, 23, 24, 25, 26, 27, 28, 29, or 30 mg quinidine; or about 50, 51, 52, 53, 54, 55, 56, 57, 58, 59, or 60 mg dextromethorphan in combination with about 20, 21, 22, 23, 24, 25, 26, 27, 28, 29, or 30 mg quinidine; in single or divided doses.

In general, the total daily dose for dextromethorphan in combination with quinidine, for symptoms associated with neurodegenerative disorders, involuntary emotional expression disorder, depression, or anxiety, or the other indications referred to herein, especially the chronic conditions, is preferably about 10 mg or less up to about 200 mg or more dextromethorphan in combination with about 1 mg or less up to about 150 mg or more quinidine. Particularly preferred total daily dosages for, e.g., depression or anxiety are about 20, 21, 22, 23, 24, 25, 26, 27, 28, 29, or 30 mg dextromethorphan in combination with about 20, 21, 22, 23, 24, 25, 26, 27, 28, 29, or 30 mg quinidine; about 30, 31, 32, 33, 34, 35, 36, 37, 38, 39, or 40 mg dextromethorphan in combination with about 20, 21, 22, 23, 24, 25, 26, 27, 28, 29, or 30 mg quinidine; about 40, 41, 42, 43, 44, 45, 46, 47, 48, 49, or 50 mg dextromethorphan in combination with about 20, 21, 22, 23, 24, 25, 26, 27, 28, 29, or 30 mg quinidine; or about 50, 51, 52, 53, 54, 55, 56, 57, 58, 59, or 60 mg dextromethorphan in combination with about 20, 21, 22, 23, 24, 25, 26, 27, 28, 29, or 30 mg quinidine; in single or divided doses. Similar daily doses for other indications as mentioned herein are generally preferred.

In managing treatment, the therapy is preferably initiated at a lower daily dose, preferably about 20 or 30 mg dextromethorphan in combination with about 2.5 mg quinidine per day, and increased up to about 60 mg dextromethorphan in combination with about 75 mg quinidine, or higher, depending on the patient's global response. It is further preferred that infants, children, patients over 65 years, and those with impaired renal or hepatic function, initially receive low doses, and that they be titrated based on individual response(s) and blood level(s). Generally, a daily dosage of 20 to 30 mg dextromethorphan and 20 to 30 mg quinidine is well-tolerated by most patients.

It can be preferred to administer dosages outside of these preferred ranges in some cases, as will be apparent to those skilled in the art. Further, it is noted that the ordinary skilled clinician or treating physician will know how and when to interrupt, adjust, or terminate therapy in consideration of individual patient response.

Any suitable route of administration can be employed for providing the patient with an effective dosage of dextromethorphan in combination with quinidine. For example, oral, rectal, transdermal, parenteral (subcutaneous, intramuscular, intravenous), intrathecal, topical, inhalable, and like forms of administration can be employed. Suitable dosage forms include tablets, troches, dispersions, suspensions, solutions, capsules, patches, and the like. Administration of medicaments prepared from the compounds described herein can be by any suitable method capable of introducing the compounds into the bloodstream. Formulations of preferred embodiments can contain a mixture of active compounds with pharmaceutically acceptable carriers or diluents as are known by those of skill in the art.

It can be advantageous to administer dextromethorphan and quinidine as an adjuvant to known therapeutic agents for the conditions to be treated according to the preferred embodiments, e.g., neurodegenerative disorders, depression, and anxiety. Antidepressants include CYMBALTA® (duloxetine); CELEXA® (citalopram); LUVOX® (fluvoxamine); PAXIL® (paroxetine); PROZAC® (fluoxetine); and ZOLOFT® (sertraline). Anti-dementia agents include but are not limited to acetylcholiesterase inhibitors, rivastigmine and donepezil. Agents for treating Parkinson's disease include but are not limited to levodopa alone or in combination with another therapeutic agent, amantadine, COMT inhibitors such as entacapone and tolcapone, dopamine agonists such as bromocriptine, pergolide, pramipexole, ropinirole, cabergoline, apomorphine and lisuride, anticholinergic mediations such as biperiden HCl, benztropine mesylate, procyclidine and trihexyphenidyl, and selegiline preparations such as Eldepryl®, Atapryl® and Carbex®. Agents for treating Alzheimer's disease include but are not limited to cholinesterase inhibitors such as donepezil, rivastigmine, galantamine and tacrine, memantine and Vitamin E. Other preferred adjuvants include pharmaceutical compositions conventionally employed in the treatment of the disorders discussed herein.

The pharmaceutical compositions of the present invention comprise dextromethorphan in combination with quinidine, or pharmaceutically acceptable salts of dextromethorphan and/or quinidine, as the active ingredient and can also contain a pharmaceutically acceptable carrier, and optionally, other therapeutic ingredients.

The terms “pharmaceutically acceptable salts” or “a pharmaceutically acceptable salt thereof” refer to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as The Merck Index. Any suitable constituent can be selected to make a salt of an active drug discussed herein, provided that it is non-toxic and does not substantially interfere with the desired activity. In addition to salts, pharmaceutically acceptable precursors and derivatives of the compounds can be employed. Pharmaceutically acceptable amides, lower alkyl esters, and protected derivatives of dextromethorphan and/or quinidine can also be suitable for use in compositions and methods of preferred embodiments. In particularly preferred embodiments, the dextromethorphan is administered in the form of dextromethorphan hydrobromide, and the quinidine is administered in the form of quinidine sulfate. For example, a dose of 30 mg dextromethorphan hydrobromide (of molecular formula C₁₈H₂₅NO·HBr·H₂O) and 30 quinidine sulfate (of molecular formula (C₂₀H₂₄N₂O₂)₂·H₂SO₄·2H₂O) may be administered (corresponding to an effective dosage of approximately 22 mg dextromethorphan and 25 mg quinidine). Other preferred dosages include, for example, 45 mg dextromethorphan hydrobromide and 30 quinidine sulfate (corresponding to an effective dosage of approximately 33 mg dextromethorphan and approximately 25 mg quinidine); 60 mg dextromethorphan hydrobromide and 30 quinidine sulfate (corresponding to an effective dosage of approximately 44 mg dextromethorphan and approximately 25 mg quinidine); 45 mg dextromethorphan hydrobromide and 45 quinidine sulfate (corresponding to an effective dosage of approximately 33 mg dextromethorphan and 37.5 mg quinidine); 60 mg dextromethorphan hydrobromide and 60 quinidine sulfate (corresponding to an effective dosage of approximately 44 mg dextromethorphan and 50 mg quinidine).

The compositions can be prepared in any desired form, for example, tables, powders, capsules, suspensions, solutions, elixirs, and aerosols. Carriers such as starches, sugars, microcrystalline cellulose, diluents, granulating agents, lubricants, binders, disintegrating agents, and the like can be used in oral solid preparations. Oral solid preparations (such as powders, capsules, and tablets) are generally preferred over oral liquid preparations. However, in certain embodiments oral liquid preparations can be preferred over oral solid preparations. The most preferred oral solid preparations are tablets. If desired, tablets can be coated by standard aqueous or nonaqueous techniques.

In addition to the common dosage forms set out above, the compounds can also be administered by sustained release, delayed release, or controlled release compositions and/or delivery devices, for example, such as those described in U.S. Pat. Nos. 3,845,770; 3,916,899; 3,536,809; 3,598,123; and 4,008,719.

Pharmaceutical compositions suitable for oral administration can be provided as discrete units such as capsules, cachets, tablets, and aerosol sprays, each containing predetermined amounts of the active ingredients, as powder or granules, or as a solution or a suspension in an aqueous liquid, a non-aqueous liquid, an oil-in-water emulsion, or a water-in-oil liquid emulsion. Such compositions can be prepared by any of the conventional methods of pharmacy, but the majority of the methods typically include the step of bringing into association the active ingredients with a carrier which constitutes one or more ingredients. In general, the compositions are prepared by uniformly and intimately admixing the active ingredients with liquid carriers, finely divided solid carriers, or both, and then, optionally, shaping the product into the desired presentation.

For example, a tablet can be prepared by compression or molding, optionally, with one or more additional ingredients. Compressed tablets can be prepared by compressing in a suitable machine the active ingredient in a free-flowing form such as powder or granules, optionally mixed with a binder, lubricant, inert diluent, surface active or dispersing agent. Molded tablets can be made by molding, in a suitable machine, a mixture of the powdered compound moistened with an inert liquid diluent.

Preferably, each tablet contains from about 30 mg to about 60 mg of dextromethorphan and from about 30 mg to about 45 mg quinidine, and each capsule contains from about 30 mg to about 60 mg of dextromethorphan and from about 30 mg to about 45 mg quinidine. Most preferably, tablets or capsules are provided in a range of dosages to permit divided dosages to be administered. For example, tablets, cachets or capsules can be provided that contain about 10 mg dextromethorphan and about 5, 10, or 15 mg quinidine; about 20 mg dextromethorphan and about 10, 20 or 30 mg quinidine; about 30 mg dextromethorphan and about 15, 30, or 45 mg quinidine; and the like. A dosage appropriate to the patient, the condition to be treated, and the number of doses to be administered daily can thus be conveniently selected. While it is generally preferred to incorporate both dextromethorphan and quinidine in a single tablet or other dosage form, in certain embodiments it can be desirable to provide the dextromethorphan and quinidine in separate dosage forms.

It has been unexpectedly discovered that patients suffering from depression, anxiety, and other conditions as described herein can treated with dextromethorphan in combination with an amount of quinidine substantially lower than the minimum amount heretofore believed to be necessary to provide a significant therapeutic effect. As used herein, a “minimum effective therapeutic amount” is that amount which provides a satisfactory degree of inhibition of the rapid elimination of dextromethorphan from the body, while producing no adverse effect or only adverse events of an acceptable degree and nature. More specifically, a preferred effective therapeutic amount is within the range of from about 20, 25 or 30 mg to about 60 mg of dextromethorphan and less than about 50 mg of quinidine per day, preferably about 20 or 30 mg to about 60 mg of dextromethorphan and about 30 mg to about 45 mg of quinidine per day, the amount being preferably administered in a divided dose based on the plasma half-life of dextromethorphan. For example, in a preferred embodiment dextromethorphan and quinidine are administered in specified mg increments to achieve a target concentration of dextromethorphan of a specified level in μg/mL plasma, with a maximum preferred specified dosage of dextromethorphan and quinidine based on body weight. The target dose is then preferably administered every 12 hours. Since the level of quinidine is minimized, the side effects observed at high dosages for quinidine are minimized or eliminated, a significant benefit over compositions containing dextromethorphan in combination with higher levels of quinidine.

It can also be desirable to use other therapeutic agents in combination with dextromethorphan. For example, it can be desirable to administer dextromethorphan in combination with a compound to treat depression or anxiety.

The compositions of the preferred embodiments, including dextromethorphan, are suitable for use in treating or alleviating symptoms of a variety of conditions, including but not limited to alcoholism (craving-withdrawal-tolerance), amyotrophic lateral sclerosis, anxiety/stress, autism, carpal tunnel syndrome, cerebral palsy, chronic cough, chronic pain, chronic obstructive pulmonary disease (COPD), dementia, agitation in dementia, depression, dermatitis, Epilepsy (e.g., pre-kindling), fibromyalgia, Huntington's disease, impotence, migraine, neuropathic pain (e.g., diabetic neuropathy, experimental wind-up pain, hyperalgesia, central summation, post-herpetic neuralgia), neuroprotection (e.g., for head injury/traumatic brain injury, ischemia, methotrexate neurotoxicity), chronic pain, pain (e.g., nociception, operative, postoperative), Parkinson's disease (e.g., motor complications with levodopa treatment), premenstrual syndrome, reflex sympathetic dystrophy, restless leg syndrome, Tourette's syndrome, voice spasm, and weaning from narcotics. The compositions of the preferred embodiments can also exhibit a neuroprotective effect (e.g., for head injury/traumatic brain injury, ischemia, methotrexate neurotoxicity), an improvement in bulbar function, and improved cognition, learning and memory (e.g., in aging).

### Pain

The compositions of preferred embodiments are effective in providing preemptive or preventative analgesia. They are typically administered prior to or during surgery, usually with anesthetics, opiates, and/or NSAIDs. Clinical trials have demonstrated that dextromethorphan decreases postoperative pain and/or analgesia consumption (opioid use), making it desirable for use in adjunctive therapy. Compositions containing dextromethorphan appear particularly effective when administered pre-operatively or peri-operatively, rather than post-operatively; however, in certain embodiments it can be desirable to administer compositions containing dextromethorphan postoperatively.

Both central sensitization after peripheral tissue injury and the development of opiate tolerance involve activation of NMDA receptors. Experimental studies have demonstrated that peripheral tissue injury may lead to hyperexcitability of nociceptive neurons in the dorsal horn, in part mediated by NMDA receptor mechanisms. Sensitization of dorsal horn neurons may be an important contributor to postoperative pain. Dextromethorphan is a weak noncompetitive NMDA receptor antagonist known to inhibit wind-up and NMDA-mediated nociceptive responses of dorsal horn neurons. Dextromethorphan inhibits spinal cord sensitization in animal models of pain and also inhibits the development of cutaneous secondary hyperalgesia after tissue trauma. NMDA studies reported reduction of nociceptive input through blockade of NMDA receptors. Tissue injury induces central sensitization in spinal cord dorsal horn neurons via mechanisms involving NMDA receptors, leading to secondary hyperalgesia. By an action on NMDA receptors, opioids also induce, in a dose dependent manner, an enhancement of this postoperative hypersensitivity. NMDA receptor antagonists enhance opioid-induced analgesia. Several drugs commonly used to treat postoperative pain, including ketamine, are linked to nitric oxide (NO) in their MOA. Biosynthesis of NO in central nervous system is tonically involved in nociceptive processing.

Nociceptive pain is pain caused by injury or disease outside the nervous system. It can be somatic or visceral, acute or chronic, and is mediated by stimulation of receptors on A-delta and C-fibers and by algogenic substances (e.g., substance P). It involves normal activation of nociceptive system by noxious stimuli. Postoperative pain and posttraumatic pain are primarily nociceptive in nature, not neuropathic.

Neuropathic pain is caused by primary lesion or dysfunction of the nervous system. It is generally chronic and highly unresponsive to traditional analgesics. Symptoms include Hyperalgesia (lowering of pain threshold and increased response to noxious stimuli) and allodynia (evocation of pain by non-noxious stimuli). Multiple pathological mechanisms underlie neuropathic pain, including peripheral and central sensitization, which results in overstimulation and hyperexcitability of nerve paths. Central sensitization, including the phenomena of wind-up (progressive increase in the number of action potentials elicited per stimulus that occurs in dorsal horn neurons due to repetitive noxious stimulation of unmyelinated C-fibers) and long-term potentiation (long lasting increase in the efficacy of synaptic transmission that may be precipitated by repetitive episodes of wind-up), involves activation of NMDA receptors.

Neuropathic pain is primarily centrally mediated pain involving a process of central sensitization. The compositions of preferred embodiments can be used to treat neuropathic conditions such as diabetic neuropathy. Studies have shown an association of NMDA receptors with development of hyperalgesia and ‘wind-up’, i.e., lasting activation of the polymodal, second-order sensory neurons in the deeper layers of the dorsal horn. Glutamate and aspartate are main neurotransmitters along ascending nociceptive pathways in the spinal cord. Glutamate, aspartate, and their receptors can be detected in particularly high concentrations in the dorsal root ganglia and the superficial laminae of the spinal cord. In low doses, glutamate receptor antagonists only slightly elevate the threshold of the physiological pain sensation. However, they suppress the process of pathological sensitization, i.e., lowering of the pain threshold seen upon excessive or lasting stimulation of C-fiber afferents, a process that takes place during inflammation or other kinds of tissue injury. At the electrophysiological level, antagonists of both the NMDA-receptors and AMPA/kainate receptors inhibit wind-up. During sensitization, the resting Mg(++) blockade of transmembrane Ca(++) channels is abolished, certain second messenger pathways are activated, the transcription of many genes is enhanced, leading to overproduction of glutamate and other excitatory neurotransmitters and expression of Na(+) channels in the primary sensory neurons activated at lower level of depolarization. This cascade of events leads to increased excitability of the pain pathways. NMDA antagonists are apparently more potent in experimental models of neuropathic pain. It is hypothesized that low-affinity NMDA channel blockers may have a better therapeutic ratio. Several clinical studies showed involvement of central sensitization mechanisms and NMDA receptor activation in mechanical allodynia/hyperalgesia and ongoing pain. NMDA receptors are involved in perception and maintenance of pathological pain in some patients. In others, pain appears to be mediated by NMDA-receptor independent mechanisms.

Temporal summation of second pain at least partly reflects temporal summation of dorsal horn neuronal responses, and both have been termed wind-up, a form of nociception-dependent central sensitization. Animal and human experiments have shown that both forms of wind-up depend on NMDA and substance P receptor systems. Wind-up of second pain in patients with fibromyalgia is enhanced compared with normal control subjects and is followed by exaggerated wind-up of second pain aftersensations and prolonged wind-up of second pain maintenance at low stimulus frequencies. Enhanced wind-up of second pain of fibromyalgia patients could be related to abnormal endogenous modulation of NDMA receptors. Central mechanisms related to referred muscle pain and temporal summation of muscular nociceptive activity are facilitated in fibromyalgia syndrome. NMDA-mediated neurotransmission may play an important role in mediating wind-up and related phenomena in pain pathways.

The compositions of preferred embodiments are efficacious in treating both nociceptive and neuropathic pain.

### Chronic Cough

Chronic cough, e.g., cough associated with cancer and respiratory infection, can also be treated using the compositions of preferred embodiments. Clinical trials demonstrated efficacy of dextromethorphan, alone or in combination therapy, for treatment of chronic cough. The antitussive effect is seemingly enhanced by quinidine in a cough model, and a subjective preference for dextromethorphan indicates a psychotropic central nervous system action. The antitussive effects of dextromethorphan were significantly and dose-dependently reduced by pretreatment with rimcazole, a specific antagonist of sigma sites. These results suggest that sigma sites may be involved in the antitussive mechanism of non-narcotic antitussive drugs. The antitussive effect dextromethorphan was also significantly reduced by pretreatment with methysergide, but not ketanserin, suggesting that 5-HT1 receptors, in particular the 5-HT1A receptors, may be more important than others for antitussive effects.

### Levodopa-Induced Motor Complications in Parkinson's Disease

The compositions of preferred embodiments are useful in treating levodopa-induced dyskinesias and spasticity. Levodopa-related motor response complications occur in most Parkinson's disease patients. Experimental evidence suggests that increased synaptic efficacy of NMDA receptors expressed on basal ganglia neurons may play a role in the pathophysiology of levodopa-induced motor response complications. Motor dysfunction produced by chronic non-physiological stimulation of dopaminergic receptors on striatal medium spiny neurons is associated with alterations in the sensitivity of glutamatergic receptors, including those of the NMDA subtype. Functional characteristics of these ionotropic receptors are regulated by their phosphorylation state. Lesioning the nigrostriatal dopamine system of rats induces Parkinsonian signs and increases the phosphorylation of striatal NMDA receptor subunits on serine and tyrosine residues. The intrastriatal administration of certain inhibitors of the kinases capable of phosphorylating NMDA receptors produces a dopaminomimetic motor response in these animals. Treating Parkinsonian rats twice daily with levodopa induces many of the characteristic features of the human motor complication syndrome and further increases the serine and tyrosine phosphorylation of specific NMDA receptor subunits. Again, the intrastriatal administration of selective inhibitors of certain serine and tyrosine kinases alleviates the motor complications. It appears that the denervation or intermittent stimulation of striatal dopaminergic receptors differentially activates signal transduction pathways in medium spiny neurons. These in turn modify the phosphorylation state of ionotropic glutamate receptors and consequently their sensitivity to cortical input. These striatal changes contribute to symptom production in Parkinson's disease. In Parkinsonism, glutamate pathways within the basal ganglia become overactive (overactive glutamatergic transmission in cortico-striatal and subthalamo-medial pallidal pathways). Thus, glutamate antagonists may possess anti-Parkinsonian qualities. Neuroleptic malignant syndrome (NMS) exhibits identical presumed pathogenesis as akinetic Parkinsonian crisis. NMDA receptor antagonists can be used for management of NMS, as these drugs are expected to exhibit hypothermic and central muscle relaxant properties.

### Learning & Memory/Cognition

Chronic organic mental disorder and autism or symptoms associated therewith can be treated by administration of the compositions of preferred embodiments. These include mental disorders associated with aging, as well as cholinergic and glutamatergic impairments. The compositions of preferred embodiments can have a beneficial effect in treating senile dementia or for cognitive enhancement in aging. The “modulatory” role of the compositions means that they exert such beneficial effects only when brain functions are perturbed. Dextromethorphan affects central nervous system serotonergic systems, the probable therapeutic mechanism. Sigma 1 ligands prevent experimental amnesia induced by muscarinic cholinergic antagonists at the learning, consolidation, or retention phase of the mnesic process. This effect involves a potentiation of acetylcholine release induced by sigma 1 ligands selectively in the hippocampal formation and cortex. Sigma 1 receptor ligands also attenuate the learning impairment induced by dizocilpine, a non-competitive antagonist of the NMDA receptor, and may relate to the potentiating effect of sigma-1 ligands on several NMDA receptor-mediated responses.

### Dementia

Symptoms of Alzheimer's disease, vascular disease, mixed dementia, and Wernicke-Korsakoff Syndrome are each amenable to treatment by administration of the compounds of preferred embodiments. Neuroprotection and cognitive improvement can be provided by administration of low affinity, noncompetitive NMDA receptor antagonists with fast open-channel blocking kinetics and strong voltage-dependency. These compositions have desirable efficacy and safety profiles. Alzheimer's disease, vascular disease, and mixed dementia (i.e., coexistence of Alzheimer's disease and vascular disease) are the three most common forms of dementia affecting older people. Alzheimer's disease is an age-related neurodegenerative disease that affects approximately 4.5 million people in the United States, as of 2005. Overstimulation of NMDA receptors by glutamate is implicated in neurodegenerative disorders, and there is increasing evidence for involvement of glutamate-mediated neurotoxicity in the pathogenesis of Alzheimer's disease. NMDA receptor-mediated glutamate excitotoxicity plays a major role in Abeta-induced neuronal death. There is a hypothesis of glutamate-induced neurotoxicity (excitotoxicity) in cerebral ischemia associated with vascular disease.

The NMDA receptor antagonist memantine may prevent excitatory neurotoxicity in dementia. Memantine acts as a neuroprotective agent in various animal models based on both neurodegenerative and vascular processes as it ameliorates cognitive and memory deficits. Memantine's mechanism of action of symptomatological improvement of cognition in animal models is unclear but might be related to an enhancement of AMPA receptor mediated neurotransmission.

NMDA receptor antagonists can be employed to inhibit the pathological functions of NMDA receptors while physiological processes in learning and memory are unaffected. The voltage-dependency of Mg++ is so pronounced that under pathological conditions it leaves the NMDA channel upon moderate depolarization, thus interrupting memory and learning. Preferably, the NMDA receptor antagonist rapidly leaves the NMDA channel upon transient physiological activation by synaptic glutamate (restoring significant signal transmission), but blocks the sustained activation of low glutamate concentration under pathological conditions, i.e., to protect against excitotoxicity as a pathomechanism of neurodegenerative disorders.

### Neuroprotection for Ischemia and Head Injury/Traumatic Brain Injury

Preclinical evidence indicates NMDA receptor antagonists such as dextromethorphan are efficacious in treating ischemia (e.g., focal cerebral ischemia) and provides neuroprotection (e.g., during cardiac surgery) and limited clinical evidence of efficacy. Excitotoxicity (excess glutamate acting on NMDA receptors) is thought to be a primary cause of delayed neuronal injury after ischemia, head injury, traumatic brain injury, spinal cord injury, hypoxia, or asphyxia. For optimum effect, the compositions of preferred embodiments are preferably administered as soon as possible after injury, or prophylactically before injury occurs.

Delayed neuronal death following hypoxic ischemic insult is primarily mediated by NMDA receptors. Brain tissue hypoxia resulted in modification of NMDA receptor ion channel and its modulatory sites. Hypoxia increased the affinity of both the ion channel and the glutamate recognition site in the immature animal. It is concluded that hypoxia-induced modification of the NMDA receptor ion channel complex leads to increased intracellular Ca(++) potentiating free radical generation and resulting in hypoxic cell injury. Asphyxia sets in, causing a progression of intracellular events which culminate in neuronal death, and this process may take up to 48 h to complete. Entry of calcium into the neuron appears to be the key to the cell death, and it is known that during asphyxia, excessive glutamate is released which stimulates the voltage-dependent NMDA receptor to open with an accumulation of excess intracellular calcium.

### Irritable Bowel Syndrome

Visceral hypersensitivity is a common feature of functional gastrointestinal disorders. One speculated mechanism is activity-dependent increase in spinal cord neuronal excitability (central sensitization), dependent on NMDA receptor activation. IBS is a common gastrointestinal disorder characterized by chronic abdominal pain and altered bowel function (diarrhea and/or constipation). Although the pathophysiology of IBS is unknown, visceral hypersensitivity (i.e., decreased pain thresholds in response to gut distension) is a biological marker of disorder. We have evidence that patients with IBS and visceral hypersensitivity also have cutaneous hypersensitivity in response to experimental thermal pain stimuli. These new findings differ from previous investigations that indicated IBS-associated hypersensitivity is limited to the gut. Rather, our data suggest that patients with IBS have alterations in central pain processing mechanisms that may represent the underlying pathophysiological basis for visceral and cutaneous hypersensitivity. Based on our preliminary data, we propose that alterations in spinal processing mechanisms are similar in patients with IBS to those that have been described for patients with other chronic pain disorders. Cutaneous hypersensitivity is also seen in other chronic pain conditions such as fibromyalgia where altered central pain processing mechanisms have been shown to be responsible for maintaining hypersensitivity. We hypothesize that IBS patients have increased peripheral and central afferent processing of nociceptive cutaneous and visceral stimuli.

### Voice Spasm

DM alters reflexes of larynx (voice box), and might change voice symptoms in people with voice disorders due to uncontrolled laryngeal muscle spasms. These include abductor spasmodic dysphonia (breathy voice breaks), adductor spasmodic dysphonia (vowel breaks), muscular tension dysphonia (tight strained voice), and vocal tremor (tremulous voice). In animal studies, dextromethorphan blocked one of reflexes in larynx that may be associated with spasms in laryngeal muscles.

### Rett Syndrome

Rett syndrome (RTT) is disorder in which nervous system does not develop properly. Rett syndrome generally affects girls, but there are some boys who have been diagnosed with Rett syndrome. Symptoms of Rett syndrome include small brain size, poor language skills, repetitive hand movements, and seizures. Recent studies demonstrate increased brain NMDA receptors in stages 2 and 3 of disease. This age-specific increase in glutamate levels and their receptors contribute to brain damage.

It can also be desirable to use other therapeutic agents in combination with dextromethorphan. For example, it can be desirable to administer dextromethorphan in combination with a compound to treat depression or anxiety.

### Depression

Clinical depression can be treated using the compositions of preferred embodiments. Interaction with the sigma-1 receptor may strengthen antidepressant effects of the compositions. For example, the NMDA receptor antagonist ketamine improved clinical postoperative and major depressive symptoms. Multicase evidence showed that that a single IV dose of this NMDA receptor antagonist provided sustained depressive symptom relief. Antidepressant-like effects of NMDA receptor antagonists in animal models implicate the glutamate system in depression and mechanism of action of antidepressants. Certain sex hormones in the brain (neurosteroids) are known to interact with sigma-1 receptors. Sigma-1 receptors regulate glutamate NMDA receptor function and the release of neurotransmitters such as dopamine. The most distinctive feature of the action of sigma-1 receptor ligands is their “modulatory” role. In behavioral studies of depression and memory, they exert beneficial effects only when brain functions are perturbed. Sigma-1 agonists modulate intracellular calcium mobilization and extracellular calcium influx, NMDA-mediated responses, acetylcholine release, and alter monoaminergic systems. A growing body of preclinical research suggests brain glutamate systems may be involved in pathophysiology of major depression and the mechanism of action of antidepressants. Antidepressant-like activity can be produced by agents that affect subcellular signaling systems linked to excitatory amino acid (EAA) receptors (e.g., nitric oxide synthase). In view of the extensive colocalization of EAA and monoamine markers in nuclei such as the locus coeruleus and dorsal raphe, it is likely that an intimate relationship exists between regulation of monoaminergic and EAA neurotransmission and antidepressant effects. There is also evidence implicating disturbances in glutamate metabolism, NMDA and NMDA, and mGluR1 and 5 receptors in depression and suicidality.

### Anxiety/Stress

Sigma receptors are closely linked to dopaminergic system. Findings suggest dysfunction in mesolimbic dopaminergic neurons is responsible for development of conditioned fear stress, and this stress response is restored through phenytoin-sensitive sigma-1 receptors, which are closely connected to dopaminergic neuronal systems. The glutamatergic system is a potential target for anxiolytic drugs. Antagonists and partial agonists of the glycine receptor inhibit function of NMDA receptor complex and evoke in animals an anxiolytic-like response.

### Ulcer

Ulcer-protective activity of sigma-receptor ligands may be related to their stimulating effect on bicarbonate secretion through interaction with sigma-receptor in the gastrointestinal mucosa.

### Migraine

Spreading depression (SD) is a profound but transient depolarization of neurons and glia that migrates across the cortical and subcortical gray at 2-5 mm/min. Under normoxic conditions, spreading depression occurs during migraine aura where it precedes migraine pain but does not damage tissue. A mechanism capable of transforming episodic to chronic migraine is attributed to hyperalgesia and related neuroplastic changes, chiefly long-term potentiation, due to action of EAAs, chiefly ones acting at NMDA receptor. A preeminent role is attributed to ‘third hyperalgesia’, newly observed which is inheritable and can act as a ground for ‘chronicization’ of migraine, while the role of primary and secondary hyperalgesia is in giving redundance to neuraxial abnormalities.

### Sleep

Normal aging is accompanied by changes in sleep-related endocrine activity: increase in cortisol at its nadir and a decrease in renin and aldosterone. More time is spent awake and slow-wave sleep is reduced: loss of sleep spindles and accordingly a loss of power in sigma frequency range. Studies showed close association between sleep architecture, especially slow-wave sleep, and activity in glutamatergic and GABAergic system. Natural NMDA antagonist and GABA(A) agonist Mg(2+) seems to play key role in regulation of sleep and endocrine systems such as HPA system and renin-angiotensin-aldosterone system (RAAS).

### Impulse Control Disorders/Compulsive Behavior

A growing body of literature implicates interactions between glutamatergic and neostriatal dopaminergic neurotransmitter systems in development and expression of impulsivity, hyperactivity, and stereotypy. Eating disorders are compulsive behavior disease, characterized by frequent recall of anorexic thoughts. Evidence suggests that memory is neocortical neuronal network, excitation of which involves hippocampus, with recall occurring by re-excitement of the same specific network. Excitement of hippocampus by NMDA receptors, leading to long-term potentiation (LTP), can be blocked by ketamine. Continuous block of long-term potentiation prevents new memory formation but does not affect previous memories. Opioid antagonists prevent loss of consciousness with ketamine but do not prevent LTP block.

### Sensorineural/Nonconductive Smell Disorders

Treatment of non-conductive olfactory disorders is to a large extent an unsolved problem. Potential mechanisms for hypothesized effect include reduced feedback inhibition in olfactory bulb as consequence of NMDA antagonistic actions and antagonism of excitotoxic action of glutamate.

### Inner Ear Tinnitus

Tinnitus is a ringing in the ears. A hypothesis of pathophysiology of inner ear tinnitus (cochlear-synaptic tinnitus) is that physiological activity of NMDA and AMPA receptors at subsynaptic membranes of inner hair cell afferents is disturbed.

### Huntington's Disease

Preclinical and clinical evidence demonstrates the efficacy NMDA-receptor antagonists for treatment of symptoms associated with Huntington's disease. NMDA receptor supersensitivity on striatal neurons may contribute to choreiform dyskinesias, and excitotoxicity may play a role in the pathogenesis of Huntington's disease. Chorea in Huntington's disease and in levodopa-induced dyskinesias of Parkinson's disease may be clinically indistinguishable.

### Alcoholism

Ethanol is a NMDA receptor antagonist and ethanol dependence upregulates NMDA receptors. Preclinical and clinical evidence indicates that NMDA receptor antagonists are effective for treating craving-withdrawal-tolerance in alcoholism. For example, acamprosate is used for relapse prophylaxis (anti-craving) in weaned alcoholics in Europe, and has been approved by the FDA for this indication in the United States. Acamprosate may impair memory functions in healthy humans, and also acts by antagonizing metabotropic glutamate receptors (mGluR5).

### Epilepsy

Epilepsy is characterized by recurrent seizures. There is excessive L-Glu release during epileptic seizures. There is growing evidence that NMDA receptor activation may play crucial role in epilepsy. EAA antagonists have anticonvulsant properties. NMDA antagonists as anticonvulsants are especially active in preventing the generalization of behavioral and electrical seizures and display a typical spectrum of in vitro antiepileptiform activities. In addition, based on in vitro and in vivo limbic kindled studies, the drugs should be regarded more as an antiepileptiform than as an anticonvulsant drugs. Dextromethorphan has antiepileptic and neuroprotective properties. However, use of dextromethorphan in these new clinical indications requires higher doses than antitussive doses, which may therefore induce phencyclidine (PCP)-like adverse events (memory and psychotomimetic disturbances) through its metabolic conversion to the active metabolite dextrorphan, a more potent PCP-like non-competitive antagonist at the NMDA receptor than dextromethorphan. Therefore, the identification of dextromethorphan metabolism phenotype, an adapted prescription, and a pharmacological modulation of the dextromethorphan metabolism may avoid adverse events. NMDA receptor antagonists including MgSO₄ and felbamate are currently used for epileptic seizures.

### Non-Ketotic Hyperglycinemia (NKH)

NKH is a rare and lethal congenital metabolic disease with autosomal recessive inheritance, causing severe, frequently lethal, neurological symptoms in the neonatal period. NKH causes muscular hypotonia, seizures, apnea, and lethargy, and it has a poor prognosis. The metabolic lesion of NKH is in the glycine cleavage system (GCS), a complex enzyme system with four enzyme components: P-, T-, H-, and L-protein. Enzymatic analysis revealed that 86% of the patients with NKH are deficient of P-protein activity. Strong GCS expression was observed in rat hippocampus, olfactory bulbus, and cerebellum. Distribution of GCS expression resembles that of NMDA receptor which has binding site for glycine. Glycine is a co-agonist of glutamate at the NMDA receptor, increasing the affinity of the receptor for the endogenous agonist glutamate. It is, therefore, suggested that the neurological disturbance in NKH may be caused by excitoneurotoxicity through the NMDA receptor allosterically activated by high concentration of glycine. Trials have been carried out with a therapy that diminishes the levels of glycine, benzoate (BZ), and another that blocks the excitatory effect in NMDA receptors (dextromethorphan).

### Toxicity

NMDA receptor antagonists such as dextromethorphan can also be employed to provide neuroprotection against methotrexate (MTX) neurotoxicity. One potential biochemical pathway for MTX neurotoxicity involves production of excitatory NMDA receptor agonists; the mechanism of action is likely multifactorial. A short course of dextromethorphan therapy was demonstrated to resolve symptoms of MTX neurotoxicity. Methotrexate-induced neurotoxicity (MTX-Ntox) is frequent complication of MTX therapy for patients with both malignant and inflammatory diseases. Methotrexate (formerly amethopterin) is an antimetabolite used in treatment of certain neoplastic diseases, severe psoriasis, and adult rheumatoid arthritis. Symptoms can present in acute, subacute, or late setting form, and can range from affective disorders, malaise, and headaches, to somnolence, focal neurological deficits, and seizures. While the pathogenesis of MTX-Ntox is likely multifactorial, one potential biochemical pathway leading from MTX to neurotoxicity involves the folate dependent remethylation of homocysteine (Hcy). MTX therapy is known to cause elevations of both plasma and CSF Hcy. Hcy is directly toxic to vascular endothelium and it and its metabolites are excitatory agonists of the NMDA receptor.

NMDA receptors in cochlea may be involved in ototoxic effects of aminoglycosides in animals. Aminoglycoside antibiotics enhance the function of NMDA receptors by interaction with a polyamine modulatory site. High doses of aminoglycosides may increase calcium entry through NMDA receptor-associated channel and promote degeneration of hair cells and cochlear nerve fibers. Organophosphorus nerve agents are considered as potential threats in both military and terrorism situations. They act as potent irreversible inhibitors of acetylcholinesterase in both central nervous system and peripheral nervous system. Numerous studies have shown that glutamate also plays a prominent role in the maintenance of organophosphate-induced seizures and in the subsequent neuropathology especially through overactivation of NMDA receptors.

### Prion Diseases

Apoptotic neuronal cell death is a hallmark of prion diseases. The apoptotic process in neuronal cells is thought to be caused by the scrapie prion protein, PrPSc, and can be experimentally induced by its peptide fragment, PrP106-126. Changes in the permeability of blood-brain barrier (BBB) and Ca(2+)-overload may participate in pathogenesis of infectious brain edema. Infectious brain edema is not only cytotoxic brain edema (intracellular edema) but also vasogenic brain edema (extracellular edema) followed by earlier blood-brain barrier breakdown, so infectious brain edema is complicated with brain edema. NMDA receptor antagonists such as dextromethorphan can also be employed to provide protection against apoptotic neuronal cell death.

### Central Nervous System Myelination in Multiple Sclerosis

Because neuronal integrity is required for central nervous system myelination, it is postulated that neuroprotective molecules, such as dextromethorphan, might favor myelination, and thus be effective in treating symptoms associated with multiple sclerosis.

### Clinical Study—Emotional Lability

A clinical study was conducted determine if a combination of dextromethorphan and quinidine was effective in suppressing or eliminating emotional lability (pseudobulbar affect) in patients with amyotrophic lateral sclerosis, multiple sclerosis or stroke.

This investigation was a randomized, double-blind, placebo-controlled, crossover, single-center study of the efficacy of oral dextromethorphan/quinidine in patients with amyotrophic lateral sclerosis, multiple sclerosis, or stroke, who were experiencing emotional lability. The 9-week study had two 4-week double-blind Treatment Periods separated by a 1-week Washout Period. Participants were randomized equally to active drug or placebo treatments. Participants were instructed to start treatment with placebo or a capsule containing 30 mg dextromethorphan combined with 75 mg quinidine. The dose was to be taken at bedtime for five consecutive days, after which a morning dose was to be added if the nighttime dose had been well tolerated. After this time the medication was to be taken at 12-hour intervals. Patients were to be treated for 4 weeks during an initial Treatment Period, after which the medication or placebo would be stopped for a 1 week Washout Period, in order to reduce the possibility of carryover effects. Thereafter, participants were to enter a second 4-week Treatment Period using active drug or placebo. To determine the effect of treatment, participants were asked to fill out an emotional lability questionnaire on the first and last day of each Treatment Period. This questionnaire was scored to measure the response to treatment.

The primary goal of this study was to determine if a combination of dextromethorphan and quinidine was effective in suppressing or eliminating emotional lability in patients with amyotrophic lateral sclerosis, multiple sclerosis, or stroke. Amyotrophic lateral sclerosis in combination with emotional lability is a severe and debilitating disease. The study was designed as a double-blind, crossover study so that each subject would be his or her own control. The two double-blind Treatment Periods were separated by a 1-week Washout Period to reduce the possibility of carryover effects. The efficacy of the treatment was determined by comparing the scores of the emotional lability questionnaire administered before and after each Treatment Period.

The protocol listed the following inclusion criteria: (1) patient had to be 20 years of age or older; (2) patient had to have a diagnosis of amyotrophic lateral sclerosis, multiple sclerosis, or stroke; (3) patient had to exhibit explosive tearfulness and/or laughter; (4) patients must have had normal hematologic, hepatic, and renal function as determined by standard laboratory tests (CBC, SMA-12, and urinalysis). The protocol specified that patients must not meet the following criteria: (1) patients whose intellectual functions were impaired sufficiently to interfere with their ability to offer informed consent or their ability to understand instructions; (2) patients with cardiac arrhythmias (AV block or prolonged QT interval), heart disease or abnormal electrocardiograms; (3) patients with known sensitivity to quinidine; (4) patients with liver, kidney or pulmonary disease; (5) patients with coexistent major systemic diseases that would interfere with interpretation of the results of the study: malignancy, poorly-controlled diabetes, ischemic cardiac disease, etc. (each patient was to be evaluated individually); (6) patients who were pregnant; (7) patients with tinnitus, optic neuritis, or myasthenia gravis; (8) all patients with prior history of major psychiatric disturbance.

The investigator could discontinue individual patients from the study at any time. Patients were encouraged to complete the study; however, they could voluntarily withdraw at any time. If a patient discontinued, the investigator provided a written report describing the reason for discontinuation. If a patient withdrew or was discontinued from the study before completion, every effort was made to complete the scheduled assessments.

During the two double-blind portions of the study, patients were randomized to receive placebo or dextromethorphan/quinidine at a total daily dose of 60 mg dextromethorphan and 150 mg quinidine. Each capsule of active drug consisted of one capsule containing 30 mg Dextromethorphan USP and 75 mg Quinidine Sulfate USP. Clinical trial material (CTM) was packaged by Bellegrove Pharmacy, Bellevue, Wash. Each dose of placebo consisted of one inert capsule. All patients were to receive two doses of CTM daily for up to 4 weeks per study period. The dose was to be taken orally at bedtime for 5 consecutive days, after which a morning dose was to be added if the nighttime dose had been well tolerated. At this time, the medication was to be taken orally at 12-hour intervals. Patients were treated for 4 weeks, after which the medication or placebo was stopped for a 1-week Washout Period. Thereafter, participants entered a second 4-week Treatment Period using active drug or placebo.

Dextromethorphan/quinidine was administrated in a randomized, double-blind, placebo-controlled, cross-over design. A clinical study coordinator randomly assigned the Treatment Period (1 or 2) in which the subject would receive dextromethorphan/quinidine. Neither the patient nor the treating physician was aware of treatment order. Subjects self-administered the dextromethorphan/quinidine capsule or placebo twice per day at 12-hour intervals for 28 consecutive days. The twice-daily dose of 30 mg dextromethorphan and 75 mg quinidine was derived from an earlier published study by Zhang et al., 1992.

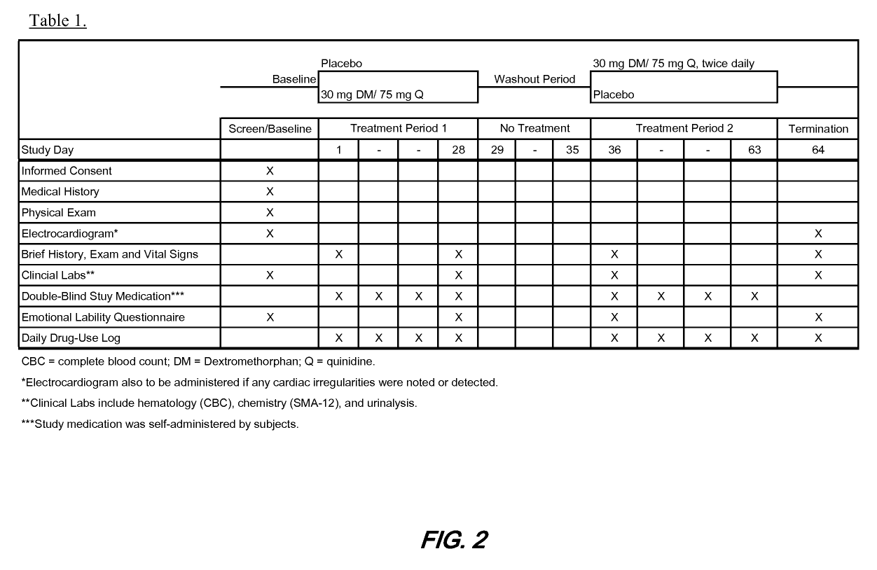

All nonessential concomitant medications were to be discontinued starting at least 1-week before the study. At the discretion of the investigator, the patient could receive medications required for the treatment of any concomitant condition or illness, with the exception of drugs known to affect emotional behavior. These exceptions included the following: sedatives, antidepressants (e.g., amitriptyline, fluoxetine), antipsychotics (e.g., fluphenazine, lithium), antianxietolytics (e.g., diazepam), hypnotics (triazolam), and drugs that affect dopamine (e.g., L-dopa, amantadine). Any drug known to be a neuromuscular blocking agent was also excluded (particularly succinylcholine, tubocurarine, and decamethonium). No other investigational products or medications were to be used by any patient during the study. Use of all medications and the reason for taking them were to be recorded. The treatment schedule is provided in Table 1 of FIG. 2.

The primary efficacy variable was a 65-item self-report measure/questionnaire that provided scores for total labile affect. This questionnaire contained 65 questions concerning the moods of the subjects. The questions were identified through interviews with ten amyotrophic lateral sclerosis patients identified by their physicians as having affective lability or loss of emotional control. Whenever possible, each patient's immediate family members were also interviewed. Responses were used to construct potential questionnaire items, which were submitted to five neurologists, familiar with both amyotrophic lateral sclerosis and affective lability, for review and suggestions. The original items measured were: labile frustration, impatience, and anger; pathological laughter; and labile tearfulness. The questions were rated on a 1-5 point scale with 1 indicating that the mood described in the question never applies, and 5 indicating that the mood described applies most of the time. All questions were phrased such that a score of 1 suggested a normal response and 5 suggested an overreactive response. These 65 items were later condensed into a 57-item questionnaire (Moore et al., 1997) and then to the 7-item Center for Neurological Study-Lability Scale (CNS-LS). The seven questions paired down from the 65-item questionnaire, eliminated any redundancies and specifically identified labile laughter and tearfulness. A response to treatment was described as a change in the total score measurement based on this emotionality-based self-reporting questionnaire. Change in the total score was used to determine the response to therapy. Efficacy in this study was assessed only during the two double-blind portions of the study.

The primary efficacy variable was a 65-item self-report measure that provided a score for total labile affect. A response to treatment was to be described as a change in the total score measurement recorded before and after Treatment Periods. This questionnaire evolved into the abbreviated 7-item self-report measure named CNS-LS used in later studies. The range of possible scores for the CNS-LS is 7 to 35. A cut-off score of 13 was selected for this scale because it provided the highest incremental validity (Moore et al., 1997) accurately predicting the neurologists' diagnoses of emotional lability for 82% of participants with a sensitivity of 0.84 and a specificity of 0.81. This questionnaire is the only validated instrument for the measurement of emotional lability for use with amyotrophic lateral sclerosis subjects.

Analyses of Efficacy Variables involved a two-treatment, two-period, two-sequence crossover design. The primary objective of this study was to determine if a combination of dextromethorphan and quinidine was effective in suppressing or eliminating emotional lability in patients with amyotrophic lateral sclerosis, multiple sclerosis, and stroke by comparing it to patients treated with placebo. The analyses of efficacy were focused primarily on changes from baseline in total score of the 65-item self-report emotional lability questionnaire. This measure provided scores for total labile affect. Change in the total score was to be used to determine the response to therapy. The analyses of treatment effect, period effect, and sequence effect were performed on the basis of the following analysis of variance (ANOVA) model: Change in total emotional lability score=effect of an overall mean+effect due to sequence+effect due to patient within sequence+effect due to period+effect due to treatment+random error It was assumed that the random error had a normal distribution. Efficacy analysis was conducted on all patients randomized to the study who received at least one dose of clinical trial material (the intent-to-treat (ITT) population). The General Linear Models procedures (PROC GLM) of the SAS® system were used to perform the statistical analyses.

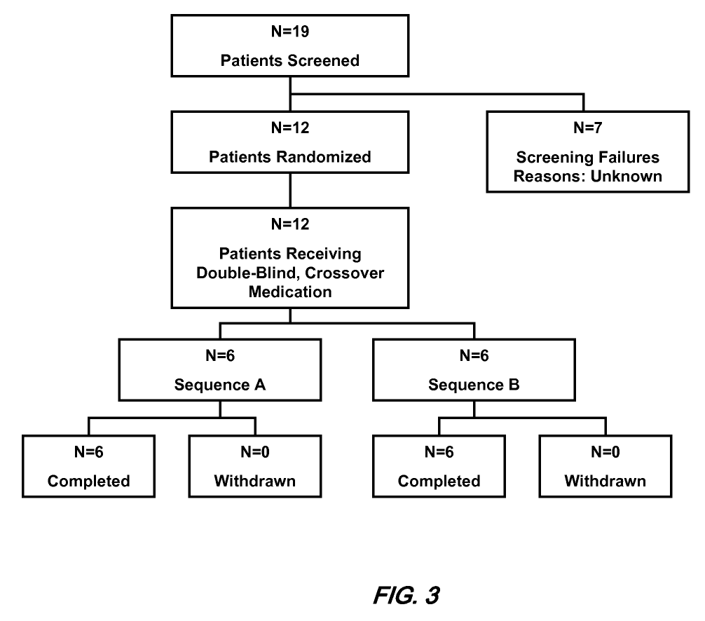

It was estimated that 22 subjects would provide a power of 80% and an a level of 0.05 to detect a significant difference in the total emotional lability score between patients receiving dextromethorphan/quinidine and patients receiving placebo. The patient distribution data are provided in FIG. 3.

The intent-to-treat population included all randomized patients who received at least one dose of clinical trial material and had a baseline measurement and at least one efficacy measurement after treatment initiation. Efficacy analyses were performed on the intent-to-treat population. The safety population included all randomized patients who received at least one dose of clinical trial material. No safety analyses were performed on the safety population because no adverse events were recorded. Characteristics of the population are provided in Table 2.

The analyses of efficacy for this study focused primarily on change in total emotional lability score from baseline to the completion of the study treatment period. The time points for evaluation by the 65-item self-reported measure were at the beginning of Treatment Period 1 (Day 1), at the end of Treatment Period 1 (Day 28), at the beginning of Treatment Period 2 (Day 36), and at the end of Treatment Period 2 (Day 65). The total emotional lability scores for each period and each sequence were summarized by descriptive statistics. Table 3 provides a summary of total emotional lability score by sequence and period.

TABLE 3

| | Mean (SD) of Total Emotional Lability Score | | | | | |
|---|---|---|---|---|---|---|
| | Treatment Period 1 | | | Treatment Period 2 | | |
| | Baseline | Treatment | Change | Baseline | Treatment | Change |
| Sequence | (N = 6) | (N = 6) | (N = 6) | (N = 6) | (N = 6) | (N = 6) |
| Sequence One | 122.5 | 98.8 | −23.7 | 115.7 | 138.2 | 22.5 |
| (DM/Q: Placebo) | (40.23) | (28.00) | (31.46) | (34.58) | (41.15) | (23.30) |
| Sequence Two | 172.8 | 170.0 | 2.8 | 161.7 | 99.8 | −61.8 |
| (Placebo: DM/Q) | (28.06) | (31.16) | (24.52) | (25.32) | (30.36) | (16.86) |

DM/Q = dextromethorphan and quinidine.

The change in total emotional lability score from baseline for each sequence was summarized by using descriptive statistics. A summary of change in total emotional lability score by sequence and treatment are provided in Table 4.

TABLE 4

| | Mean (SD) of Change in Total Emotional Lability Score | | |
|---|---|---|---|
| | Change from Baseline | | Difference between |
| | DM/Q | Placebo | DM/Q and Placebo |
| Sequence | (N = 6) | (N = 6) | (N = 6) |
| Sequence One (DM/Q: Placebo) | −23.7 (31.46) | 22.5 (23.30) | −46.2 (34.18) |
| Sequence Two (Placebo: DM/Q) | −61.8 (16.86) | −2.8 (24.52) | −59.00 (30.07) |

DM/Q = dextromethorphan and quinidine.

An ANOVA model was used to analyze the treatment effect, the period effect, and the sequence effect on changes in total emotional lability score from baseline. The results are presented in Table 5. There was no statistically significant period effect. The treatment effect and sequence effects were statistically significant.

In accordance with the protocol, the primary analysis of the change in total emotional lability score from baseline was performed on the intent-to-treat population. An ANOVA model was used to analyze the treatment effect and period effect. The results demonstrated that there was a statistically significant treatment effect (p=0.0001) and that there was no statistically significant period effect (p=0.5299).

The primary objective of this single-center Phase 2 study was to determine if a combination of dextromethorphan and quinidine was effective in treating emotional lability (pseudobulbar affect) in patients with neurodegenerative disease/disorder (including amyotrophic lateral sclerosis, multiple sclerosis, or stroke). The study was designed as a double-blind, cross-over, placebo-controlled study. Patients were randomized into two groups in a 1:1 ratio to receive either active drug or placebo. The 9-week study had two 4-week double-blind Treatment Periods separated by a 1-week Washout Period. Previous research had indicated that achieving a high concentration of dextromethorphan in patients diagnosed with emotional lability provided symptomatic relief and consequently improved quality of life. The primary objective with this study was to establish the efficacy of administering dextromethorphan and quinidine in treating emotional lability in patients with certain neurological diseases/disorders. The cross-over design of the study allowed for the patients to be their own controls. By comparing the total score of the emotional lability questionnaire before and after a double-blind Treatment Period, it was possible to determine the effect of active drug versus placebo.

Even though this was a small study (N=12), it is clear from the data presented in Table 5 that the drug is active compared to placebo. This highly statistically significant result (p=0.0001) demonstrates that this novel combination of dextromethorphan and quinidine is an effective way of treating a severe and debilitating symptom of a life-threatening disease. This combination seems to be well tolerated and safe without any major adverse side effects, because no treatment-emergent adverse events were reported during the study. (There were no deaths, serious adverse events, or discontinuations during the study.) The combination of dextromethorphan and quinidine was statistically significant effective in treating emotional lability (pseudobulbar affect) in patients with amyotrophic lateral sclerosis.

### Clinical Study—Anger/Frustration/Upset

Results of the self-report measure/questionnaire were analyzed in to determine efficacy of dextromethorphan and quinidine in treating anger, frustration, upset, and combinations thereof as manifestations of emotional lability. Efficacy was determined by examining results obtained for questions specific to anger, frustration, and upset. The data, as provided in Table 6, demonstrates the effectiveness of dextromethorphan and quinidine in treating anger, frustration, upset as manifestations of emotional lability.

TABLE 6

| CNS-LS Subset | | | | | | | | | | |
|---|---|---|---|---|---|---|---|---|---|---|
| (Question Numbers) | | Baseline | | Post-treatment | | Change | | Percent Change | | P-value [1] |
| CNS-LS | N | 12 | | 12 | | 12 | | 12 | | |
| (38, 28, 36, 31, 32, 61, 35) | Mean (sd) | 17.8 | (5.3) | 11.1 | (4.1) | −6.7 | (7.5) | −29.4 | (42.6) | 0.0108 |
| | Median (min, max) | 16.0 | (9, 30) | 9.5 | (7, 20) | −7.0 | (−20, 7) | −43.8 | (−67, 78) | |
| Anger | N | 12 | | 12 | | 12 | | 12 | | |
| (1, 2, 7, 11, 20, 27, 41, | Mean (sd) | 19.7 | (7.3) | 15.7 | (4.8) | −4.0 | (4.9) | −16.4 | (18.8) | 0.0158 |
| 42, 47, 50, 52, 54) | Median (min, max) | 18.5 | (12, 33) | 13.0 | (12, 25) | −3.0 | (−13, 2) | −16.5 | (−43, 9) | |
| Frustration | N | 12 | | 12 | | 12 | | 12 | | |
| (4, 5, 8, 6, 12, 15, 29) | Mean (sd) | 16.3 | (5.3) | 10.8 | (3.1) | −5.4 | (3.4) | −30.3 | (15.3) | 0.0002 |
| | Median (min, max) | 18.0 | (7, 25) | 11.0 | (7, 17) | −6.0 | (−12, 0) | −33.3 | (−48, 0) | |
| Anger + Frustration | N | 12 | | 12 | | 12 | | 12 | | |
| | Mean (sd) | 35.9 | (10.8) | 26.5 | (7.4) | −9.4 | (6.9) | −23.7 | (16.1) | 0.0006 |
| | Median (min, max) | 35.5 | (19, 54) | 24.5 | (19, 41) | −10.5 | (−21, 1) | −28.9 | (−43, 5) | |
| Anger + Frustration + Upset | N | 12 | | 12 | | 12 | | 12 | | |
| (10, 13, 17, 30, 34, 39, | Mean (sd) | 58.5 | (17.4) | 41.8 | (11.9) | −16.7 | (12.3) | −25.9 | (16.7) | 0.0006 |
| 44, 55, 58, 60) | Median (min, max) | 62.5 | (30, 84) | 39.5 | (29, 64) | −17.5 | (−32, 1) | −27.9 | (−44, 2) | |
| Smith's auxiliary subscale | N | 12 | | 12 | | 12 | | 12 | | |
| (39, 30, 5, 7, 6, 15, 21, 50) | Mean (sd) | 16.7 | (5.7) | 11.8 | (3.6) | −4.9 | (4.2) | −24.9 | (22.1) | 0.0019 |
| | Median (min, max) | 19.5 | (8, 23) | 10.5 | (8, 19) | −5.5 | (−12, 1) | −31.1 | (−57, 13) | |

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

1. A method for treating depression, comprising: administering from about 10 mg to about 200 mg of dextromethorphan per day in combination with from about 1 mg to less than about 50 mg of quinidine per day to a patient in need thereof.

2. The method of claim 1, wherein from about 10 mg to about 45 mg of quinidine is administered per day, and wherein from about 20 mg to about 60 mg of dextromethorphan is administered per day.

3. The method of claim 1, wherein the quinidine is in a form of quinidine sulfate and wherein the dextromethorphan is in a form of dextromethorphan hydrobromide.

4. The method of claim 1, wherein quinidine and dextromethorphan are administered in a unit dosage form comprising about 45 mg dextromethorphan hydrobromide and about 10 mg quinidine sulfate.

5. The method of claim 1, wherein quinidine and dextromethorphan are administered in a unit dosage form comprising about 30 mg dextromethorphan hydrobromide and about 10 mg quinidine sulfate.

6. The method of claim 1, wherein quinidine and dextromethorphan are administered in a unit dosage form comprising about 20 mg dextromethorphan hydrobromide and about 10 mg quinidine sulfate.

7. The method of claim 1, wherein quinidine and dextromethorphan are administered in a unit dosage form comprising about 15 mg dextromethorphan hydrobromide and about 10 mg quinidine sulfate.

8. The method of claim 1, wherein quinidine and dextromethorphan are administered in a unit dosage form comprising about 10 mg dextromethorphan hydrobromide and about 10 mg quinidine sulfate.

9. The method of claim 1, wherein quinidine and dextromethorphan are administered in a unit dosage form configured for administration once a day, twice a day, or three times a day.

10. The method of claim 1, wherein quinidine and dextromethorphan are administered in a tablet unit dosage form or a capsule unit dosage form.

11. A method for treating anxiety, comprising:
administering from about 10 mg to about 200 mg of dextromethorphan per day in combination with from about 1 mg to less than about 50 mg of quinidine per day to a patient in need thereof.

12. The method of claim 11, wherein from about 10 mg to about 45 mg of quinidine is administered per day, and wherein from about 20 mg to about 60 mg of dextromethorphan is administered per day.

13. The method of claim 11, wherein the quinidine is in a form of quinidine sulfate and wherein the dextromethorphan is in a form of dextromethorphan hydrobromide.

14-22. (canceled)

23. A method for treating symptoms of multiple sclerosis, comprising:
administering dextromethorphan in combination with quinidine to a patient in need thereof, wherein the amount of dextromethorphan administered comprises from about 20 mg/day to about 60 mg/day and the amount of quinidine administered comprises from about 20 mg/day to about 45 mg/day.
